(19) United States
(12) Patent Application Publication
(10) Pub. No.: US 2022/0241072 A1
(43) Pub. Date: Aug. 4, 2022

(54) CARDIAC RESTORATION AND RESHAPING DEVICES AND METHODS

(71) Applicant: Edwards Lifesciences Corporation, Irvine, CA (US)

(72) Inventors: Bingquan Su, Irvine, CA (US); Nima V. Nia, Mission Viejo, CA (US); Amanda Tjipta Sugijoto, San Ramon, CA (US); Hengchu Cao, Irvine, CA (US); Sakyasingh Tripathy, San Diego, CA (US); Douglas Thomas Dominick, Irvine, CA (US)

(21) Appl. No.: 17/723,266

(22) Filed: Apr. 18, 2022

Related U.S. Application Data
(63) Continuation of application No. PCT/US2020/055236, filed on Oct. 12, 2020.
(60) Provisional application No. 62/926,258, filed on Oct. 25, 2019.

(51) Int. Cl. A61F 2/24; A61B 17/04

(52) U.S. Cl. CPC A61F 2/2457; A61B 2017/0464; A61B 17/0401; A61F 2/246

(57) ABSTRACT

A method comprises anchoring a first suture to a first papillary muscle, anchoring the first suture to a ventricle wall, and cinching the first suture to cause the first papillary muscle to move closer to the ventricle wall.

### RELATED APPLICATION

This application is a continuation application of PCT International Application Serial No. PCT/US2020/055236, filed Oct. 12, 2020 and entitled CARDIAC RESTORATION AND RESHAPING DEVICES AND METHODS, which claims priority based on U.S. Provisional Patent Application Ser. No. 62/926,258, filed Oct. 25, 2019 and entitled CARDIAC RESTORATION AND RESHAPING DEVICES AND METHODS, the complete disclosures of both of which are hereby incorporated by reference in their entireties.

### BACKGROUND

The present disclosure generally relates to the field of improving heart performance.

Heart Failure with reduced Ejection Fraction (HFrEF), also known as systolic heart failure, is characterized by an inability of the heart to contract adequately, resulting in less oxygen-rich blood being expelled into the body. Functional mitral valve regurgitation (FMR) is a disease that occurs when the left ventricle of the heart is distorted or dilated, displacing the papillary muscles that support the two valve leaflets. When the valve leaflets can no longer come together to close the annulus, blood may flow back into the atrium.

### SUMMARY

For purposes of summarizing the disclosure, certain aspects, advantages and novel features have been described herein. It is to be understood that not necessarily all such advantages may be achieved in accordance with any particular embodiment. Thus, the disclosed embodiments may be carried out in a manner that achieves or optimizes one advantage or group of advantages as taught herein without necessarily achieving other advantages as may be taught or suggested herein.

Some implementations of the present disclosure relate to a method comprising anchoring a first suture to a first papillary muscle, anchoring the first suture to a ventricle wall, and cinching the first suture to cause the first papillary muscle to move towards the ventricle wall.

The method may further comprise anchoring the first suture to a second papillary muscle. Cinching the first suture may cause the second papillary muscle to move towards the ventricle wall. In some embodiments, cinching the first suture causes the first papillary muscle to move towards the second papillary muscle.

A first end and a second end of the first suture may be anchored to the ventricle wall and anchoring the first suture to the first papillary muscle and the second papillary muscle may involve wrapping the first suture at least partially around the first papillary muscle and the second papillary muscle.

In some embodiments, the method further comprises anchoring a second suture to the first papillary muscle. Anchoring the first suture to the first papillary muscle may involve attaching the first suture to the second suture.

In some embodiments, the method further comprises anchoring the second suture to a second papillary muscle. Cinching the first suture may involve pulling the second suture towards the ventricle wall.

The method may further comprise anchoring a second suture to a second papillary muscle, anchoring the second suture to the ventricle wall, and cinching the second suture. Cinching the second suture may cause the second papillary muscle to move towards the ventricle wall.

In some embodiments, the first suture and the second suture at least partially overlap. The first suture may be anchored to a first portion of the ventricle wall and the second suture may be anchored to a second portion of the ventricle wall.

The first portion of the ventricle wall may be closer to the second papillary muscle than the second portion of the ventricle wall.

Some implementations of the present disclosure relate to a suture assembly comprising a first branch configured to anchor to a first papillary muscle, anchor to a ventricle wall, and cinch to cause the first papillary muscle to move towards the ventricle wall.

The first branch may be further configured to anchor to a second papillary muscle. In some embodiments, cinching the first branch further causes the second papillary muscle to move towards the ventricle wall.

Cinching the first branch may further cause the first papillary muscle to move towards the second papillary muscle. In some embodiments, a first end and a second end of the first branch are configured to be anchored to the ventricle wall.

Anchoring the first branch to the first papillary muscle and the second papillary muscle may involve wrapping the first branch at least partially around the first papillary muscle and the second papillary muscle.

In some embodiments, the suture assembly further comprises a second branch configured to anchor to the first papillary muscle. Anchoring the first branch to the first papillary muscle may involve attaching the first branch to the second branch.

In some embodiments, the second branch is configured to anchor to a second papillary muscle. The suture assembly may further comprise a second branch configured to anchor to a second papillary muscle, anchor to the ventricle wall, and cinch to cause the second papillary muscle to move towards the ventricle wall.

Some implementations of the present disclosure relate to a cardiac device comprising a suture assembly including a first branch and an anchoring element configured to anchor to a ventricle wall. The first branch is configured to attach to the anchoring element, anchor to a first papillary muscle, and cinch to cause the first papillary muscle to move towards the ventricle wall.

### BRIEF DESCRIPTION OF THE DRAWINGS

Various embodiments are depicted in the accompanying drawings for illustrative purposes, and should in no way be interpreted as limiting the scope of the inventions. In addition, various features of different disclosed embodiments can be combined to form additional embodiments, which are part of this disclosure. Throughout the drawings, reference numbers may be reused to indicate correspondence between reference elements.

FIG. 1 provides a cross-sectional view of a human heart.

FIG. 2A provides a cross-sectional view of the left ventricle and left atrium of an example heart.

FIG. 2B provides an overhead view (e.g., as viewed from the left atrium) of a mitral valve of a heart in a healthy condition.

FIG. 3A provides a cross-sectional view of a heart experiencing mitral regurgitation.

FIG. 3B provides an overhead view of a mitral valve (e.g., as viewed from the left atrium) in which the heart and/or left ventricle is in a state in which mitral regurgitation is present.

FIG. 4A provides an overhead view of an example device for correcting mitral regurgitation and/or papillary muscle displacement in accordance with one or more embodiments.

FIG. 4B provides a side cross-sectional view of the left ventricle with an implanted papillary muscle restoration device in accordance with one or more embodiments.

FIG. 5A provides an overhead view of a mitral valve showing a relative position of a restoration device implanted within the left ventricle (e.g., below the mitral valve) in accordance with one or more embodiments.

FIG. 5B provides a side view of a left ventricle with an implanted restoration device in accordance with one or more embodiments.

FIG. 6A provides an overhead view of the mitral valve (e.g., as viewed from the left atrium) showing respective positions of the papillary muscles and a restoration device implanted in the left ventricle according to one or more embodiments.

FIG. 6B provides a side view of the left ventricle with an implanted restoration device according to one or more embodiments.

FIG. 7 (FIGS. 7-1 and 7-2) provides a flow chart for a transcatheter process for delivering a restoration device to a heart according to one or more embodiments.

FIG. 8 (FIGS. 8-1 and 8-2) provides cross-sectional views of the heart for various steps of the transfemoral process of FIG. 7 according to one or more embodiments.

FIG. 9 (FIGS. 9-1 and 9-2) provides overhead views of the mitral valve including respective positions of the papillary muscles and restoration device for various stages of the flow chart represented in FIG. 7 according to one or more embodiments disclosed herein.

FIG. 10 (FIGS. 10-1 and 10-2) provides a flowchart for a transcatheter process for delivering a shaping device to a heart and specifically to a chamber of the heart (e.g., the left ventricle) in accordance with one or more embodiments.

FIG. 11 (FIGS. 11-1 and 11-2) provides cross-sectional side views of a heart for various steps of the process of FIG. 10 in accordance with one or more embodiments.

FIG. 12 (FIGS. 12-1 and 12-2) provides cross-sectional overhead views of a mitral valve and respective positions of papillary muscles and restoration devices for various steps of the process of FIG. 10 in accordance with one or more embodiments.

FIG. 13 (FIGS. 13-1, 13-2, and 13-3) provides a flowchart for a transcatheter process of delivering a restoration device comprising a suture assembly to the heart in accordance with one or more embodiments.

FIG. 14 (FIGS. 14-1, 14-2, and 14-3) provides several images corresponding to the process of FIG. 13 in which the restoration device is delivered via a transfemoral process in accordance with one or more embodiments.

FIG. 15 (FIGS. 15-1, 15-2, and 15-3) provides several overhead images of the mitral valve and reference locations of the papillary muscles with corresponding to various steps of the process of FIG. 13 in accordance with one or more embodiments.

### DETAILED DESCRIPTION

The headings provided herein are for convenience only and do not necessarily affect the scope or meaning of the claimed invention.

Although certain preferred embodiments and examples are disclosed below, inventive subject matter extends beyond the specifically disclosed embodiments to other alternative embodiments and/or uses and to modifications and equivalents thereof. Thus, the scope of the claims that may arise herefrom is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

In humans and other vertebrate animals, the heart generally comprises a muscular organ having four pumping chambers, wherein the flow thereof is at least partially controlled by various heart valves, namely, the aortic, mitral (or bicuspid), tricuspid, and pulmonary valves. The valves may be configured to open and close in response to a pressure gradient present during various stages of the cardiac cycle (e.g., relaxation and contraction) to at least partially control the flow of blood to a respective region of the heart and/or to blood vessels (e.g., pulmonary, aorta, etc.).

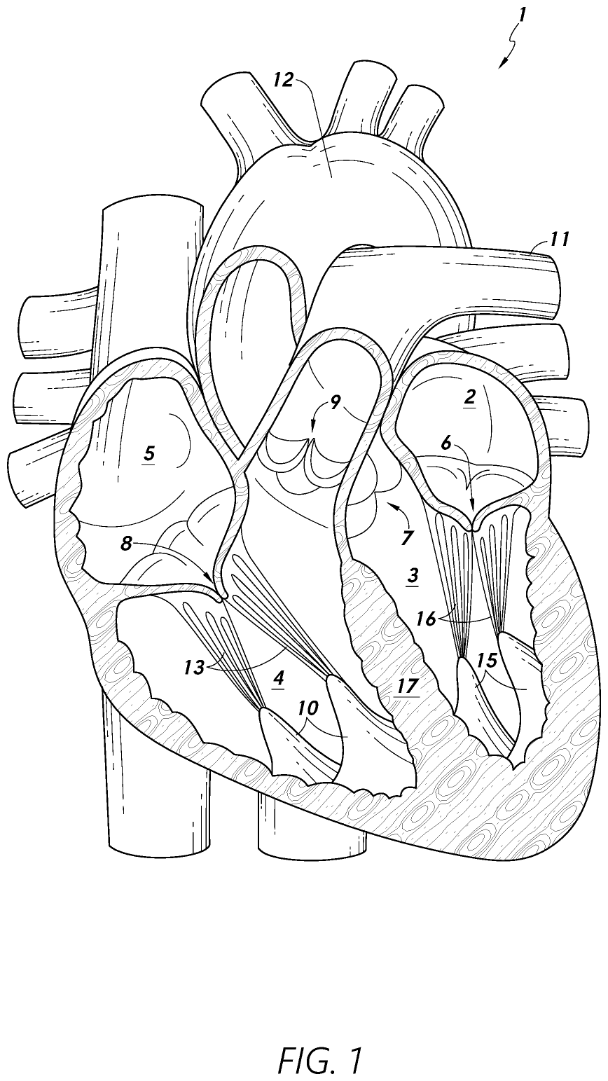

FIG. 1 illustrates an example representation of a heart 1 having various features relevant to certain embodiments of the present inventive disclosure. The heart 1 includes four chambers, namely the left atrium 2, the left ventricle 3, the right ventricle 4, and the right atrium 5. A wall of muscle 17, referred to as the septum, separates the left 2 and right 5 atria and the left 3 and right 4 ventricles. The inferior tip 19 of the heart 1 is referred to as the apex (or apex region) and is located on the midclavicular line, in the fifth intercostal space.

The heart 1 further includes four valves for aiding the circulation of blood therein, including the tricuspid valve 8, which separates the right atrium 5 from the right ventricle 4. The tricuspid valve 8 may generally have three cusps or leaflets and may generally close during ventricular contraction (i.e., systole) and open during ventricular expansion (i.e., diastole). The valves of the heart 1 further include the pulmonary valve 9, which separates the right ventricle 4 from the pulmonary artery 11, and may be configured to open during systole so that blood may be pumped toward the lungs, and close during diastole to prevent blood from leaking back into the heart from the pulmonary artery. The pulmonary valve 9 generally has three cusps/leaflets, wherein each one may have a crescent-type shape. The heart 1 further includes the mitral valve 6, which generally has two cusps/leaflets and separates the left atrium 2 from the left ventricle 3. The mitral valve 6 may generally be configured to open during diastole so that blood in the left atrium 2 can flow into the left ventricle 3, and advantageously close during diastole to prevent blood from leaking back into the left atrium 2. The aortic valve 7 separates the left ventricle 3 from the aorta 12. The aortic valve 7 is configured to open during systole to allow blood leaving the left ventricle 3 to enter the aorta 12, and close during diastole to prevent blood from leaking back into the left ventricle 3.

Heart valves may generally comprise a relatively dense fibrous ring, referred to herein as the annulus, as well as a plurality of leaflets or cusps attached to the annulus. Generally, the size of the leaflets or cusps may be such that when the heart contracts the resulting increased blood pressure produced within the corresponding heart chamber forces the leaflets at least partially open to allow flow from the heart chamber. As the pressure in the heart chamber subsides, the pressure in the subsequent chamber or blood vessel may become dominant, and press back against the leaflets. As a result, the leaflets/cusps come in apposition to each other, thereby closing the flow passage.

The atrioventricular (i.e., mitral and tricuspid) heart valves may further comprise a collection of chordae tendineae and papillary muscles for securing the leaflets of the respective valves to promote and/or facilitate proper coaptation of the valve leaflets and prevent prolapse thereof. The papillary muscles, for example, may generally comprise finger-like projections from the ventricle wall. With respect to the tricuspid valve 8, the normal tricuspid valve may comprise three leaflets (two shown in FIG. 1) and three corresponding papillary muscles 10 (two shown in FIG. 1). The leaflets of the tricuspid valve may be referred to as the anterior, posterior and septal leaflets, respectively. The valve leaflets are connected to the papillary muscles 10 by the chordae tendineae 13, which are disposed in the right ventricle 4 along with the papillary muscles 10. Although tricuspid valves are described herein as comprising three leaflets, it should be understood that tricuspid valves may occur with two or four leaflets in certain patients and/or conditions; the principles relating to papillary muscle repositioning disclosed herein are applicable to atrioventricular valves having any number of leaflets and/or papillary muscles associated therewith.

The right ventricular papillary muscles 10 originate in the right ventricle wall, and attach to the anterior, posterior and septal leaflets of the tricuspid valve, respectively, via the chordae tendineae 13. The papillary muscles 10 of the right ventricle 4 may have variable anatomy; the anterior papillary may generally be the most prominent of the papillary muscles. The papillary muscles 10 may serve to secure the leaflets of the tricuspid valve 8 to prevent prolapsing of the leaflets into the right atrium 5 during ventricular systole. Tricuspid regurgitation can be the result of papillary dysfunction or chordae rupture.

With respect to the mitral valve 6, a normal mitral valve may comprise two leaflets (anterior and posterior) and two corresponding papillary muscles 15. The papillary muscles 15 originate in the left ventricle wall and project into the left ventricle 3. Generally, the anterior leaflet may cover approximately two-thirds of the valve annulus. Although the anterior leaflet covers a greater portion of the annulus, the posterior leaflet may comprise a larger surface area in certain anatomies.

The valve leaflets of the mitral valve 6 may be prevented from prolapsing into the left atrium 2 by the action of the chordae tendineae 16 tendons connecting the valve leaflets to the papillary muscles 15. The relatively inelastic chordae tendineae 16 are attached at one end to the papillary muscles 15 and at the other to the valve leaflets; chordae tendineae from each of the papillary muscles 15 are attached to a respective leaflet of the mitral valve 6. Thus, when the left ventricle 3 contracts, the intraventricular pressure forces the valve to close, while the chordae tendineae 16 keep the leaflets coapting together and prevent the valve from opening in the wrong direction, thereby preventing blood to flow back to the left atrium 2. The various chords of the chordae tendineae may have different thicknesses, wherein relatively thinner chords are attached to the free leaflet margin, while relatively thicker chords (e.g., strut chords) are attached farther away from the free margin.

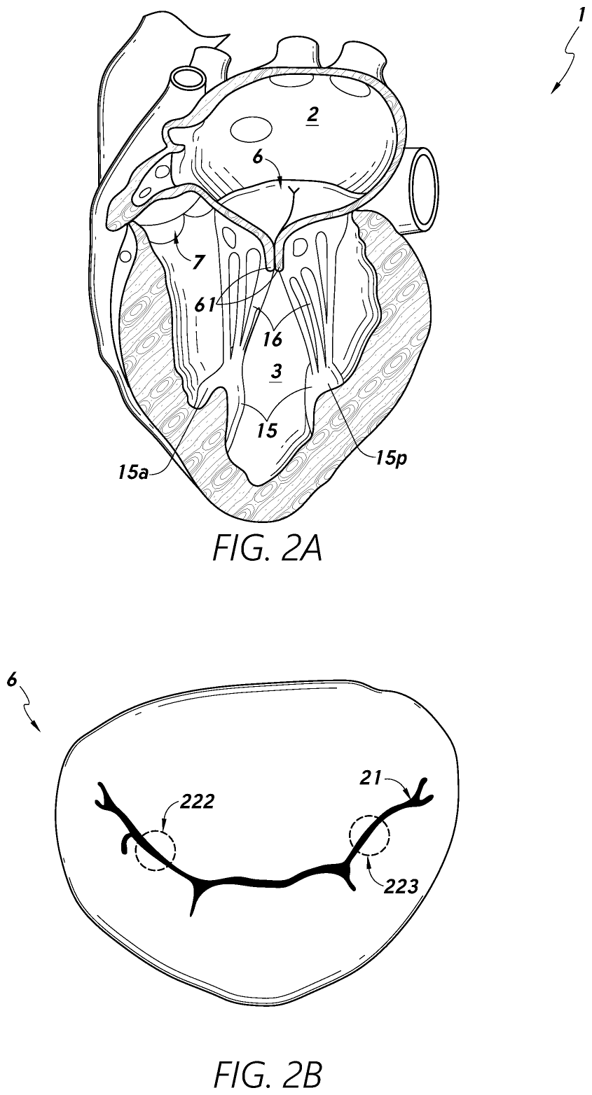

FIG. 2A provides a cross-sectional view of the left ventricle 3 and left atrium 2 of an example heart 1. While some embodiment devices and/or methods are described herein with respect to the left ventricle 3, mitral valve 6, and/or left atrium 2, such devices and/or methods may be applied to and/or performed within other areas of the heart, including the right ventricle, right atrium, and/or tricuspid valve. The diagram of FIG. 2A shows the mitral valve 6, wherein the disposition of the valve 6, papillary muscles 15 and/or chordae tendineae 16 may be illustrative as providing for proper coapting of the valve leaflets to advantageously at least partially prevent regurgitation and/or undesirable flow into the left atrium from the left ventricle 3 and vice versa. Although a mitral valve 6 is shown in FIG. 2A and various other figures provided herewith and described herein in the context of certain embodiments of the present disclosure, it should be understood that papillary muscle repositioning principles disclosed herein may be applicable with respect to any atrioventricular valve and associated anatomy (e.g., papillary muscles, chordae tendineae, ventricle wall, etc.), such as the tricuspid valve.

As described above, with respect to a healthy heart valve as shown in FIG. 2A, the valve leaflets 61 may extend inward from the valve annulus and come together in the flow orifice to permit flow in the outflow direction (e.g., the downward direction in FIG. 2A) and prevent backflow or regurgitation toward the inflow direction (e.g., the upward direction in FIG. 2A). For example, during atrial systole, blood flows from the atria 2 to the ventricle 3 down the pressure gradient, resulting in the chordae tendineae 16 being relaxed due to the atrioventricular valve 6 being forced open. When the ventricle 3 contracts during ventricular systole, the increased blood pressures in both chambers may push the valve 6 closed, preventing backflow of blood into the atria 2. Due to the lower blood pressure in the atria compared to the ventricles, the valve leaflets may tend to be drawn toward the atria. The chordae tendineae 16 can serve to tether the leaflets and hold them in a closed position when they become tense during ventricular systole. The papillary muscles 15 provide structures in the ventricles for securing the chordae tendineae 16 and therefore allowing the chordae tendineae 16 to hold the leaflets in a closed position. The papillary muscles 15 may include a first papillary muscle 15a (e.g., an anterolateral papillary muscle, which may be primarily tethered to the anterior leaflet, for example) and a second papillary muscle 15p (e.g., the posteromedial papillary muscle, which may be primarily tethered to the posterior leaflet, for example). Each of the first papillary muscle 15a and second papillary muscle 15p may provide chordae tendineae 16 to each valve leaflet (e.g., the anterior and posterior leaflets). With respect to the state of the heart 1 shown in FIG. 2A, the proper coaptation of the valve leaflets, which may be due in part to proper position of the papillary muscles 15, may advantageously result in mitral valve operation substantially free of leakage.

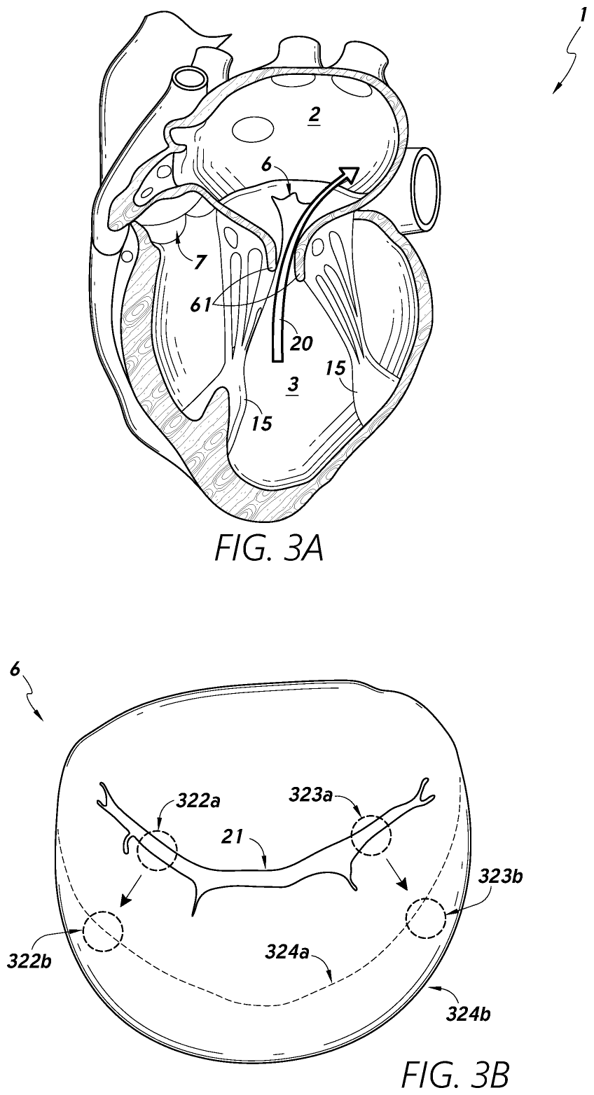

Heart valve disease represents a condition in which one or more of the valves of the heart fails to function properly. Diseased heart valves may be categorized as stenotic, wherein the valve does not open sufficiently to allow adequate forward flow of blood through the valve, and/or incompetent, wherein the valve does not close completely, causing excessive backward flow of blood through the valve when the valve is closed. In certain conditions, valve disease can be severely debilitating and even fatal if left untreated. With regard to incompetent heart valves, over time and/or due to various physiological conditions, the position of papillary muscles may become altered, thereby potentially contributing to valve regurgitation. For example, as shown in FIG. 3A, which illustrates a cross-sectional view of a heart 1 experiencing mitral regurgitation flow 20, dilation of the left ventricle may cause changes in the position of the papillary muscles 15 that allow flow 20 back from the ventricle 3 to the atrium 2. Dilation of the left ventricle can be caused by any number of conditions, such as focal myocardial infarction, global ischemia of the myocardial tissue, or idiopathic dilated cardiomyopathy, resulting in alterations in the geometric relationship between papillary muscles and other components associated with the valve(s) that can cause valve regurgitation. Functional regurgitation may further be present even where the valve components may be normal pathologically, yet may be unable to function properly due to changes in the surrounding environment. Examples of such changes include geometric alterations of one or more heart chambers and/or decreases in myocardial contractility. In any case, the resultant volume overload that exists as a result of an insufficient valve may increase chamber wall stress, which may eventually result in a dilatory effect that causes papillary muscle alteration resulting in valve dysfunction and degraded cardiac efficiency.

The positions of the papillary muscles 15 with respect to the mitral valve 6 may be of great significance in the functioning of the mitral valve 6. Because the chordae tendineae 16 connect to the papillary muscles 15 (specifically, at tips of the papillary muscles 15), migration of the papillary muscles causes corresponding migration of the chordae tendineae 16, which may cause undesirable force on the leaflets of the mitral valve. For example, as the papillary muscles migrate further apart from each other and/or further from a position below the mitral valve 6, the chordae tendineae 16 may pull the leaflets apart such that the leaflets cannot fully coapt.

FIG. 2B provides an overhead view (e.g., as viewed from the left atrium) of a mitral valve 6 of a heart in a healthy condition. For illustrative purposes, the relative positions of two papillary muscles with respect to the mitral valve 6 are represented by dashed circles. A position of a papillary muscle 222, 223 may be indicative of a tip portion of the papillary muscle (e.g., a portion of the papillary muscle that is connected to the chordae tendineae). As shown in FIG. 2B, one or more papillary muscles 222, 223 may be oriented below at least a portion of the mitral valve 6. While FIG. 2B shows positions of two papillary muscles 222, 223, a ventricle may include any number of papillary muscles. In some cases, the papillary muscles may be positioned directly below or nearly directly below the coaptation line 21 between leaflets and/or portions of leaflets of the mitral valve 6 and/or other valve of the heart. However, the papillary muscles 222, 223 may be positioned with different orientations with respect to the mitral valve in some cases. The general positions of the papillary muscles (particularly the tips of the papillary muscles) can have significant effect on the opening and closing of the mitral valve 6. In some cases, a heart not experiencing dilation may be oriented such that the papillary muscles may be located directly below or near nearly directly below the coaptation line of the mitral valve. As the heart and/or ventricle dilates, one or more papillary muscles may move away from the coaptation line 21 of the mitral valve and/or a central area of the mitral valve. (See, e.g., FIG. 3B). Dilation of a heart may cause and/or may be characterized by stretching and/or thinning of the walls of the heart. Consequently, an inner volume of one or more chambers of the heart may increase. In some cases, as the papillary muscles move away from a central portion of the mitral valve 6, the chordae tendineae 16 tethered between the papillary muscles and the mitral valve 6 may cause the mitral valve 6 to open and/or may prevent the mitral valve 6 from closing.

FIG. 3A provides an illustration of the heart 1 in a state where functional mitral valve regurgitation (FMR) is present. FMR may be considered a disease of the left ventricle 3, rather than of the mitral valve 6. For example, mitral valve regurgitation may occur when the left ventricle 3 of the heart 1 is distorted or dilated, displacing the papillary muscles 15 that support the two valve leaflets 61. The valve leaflets 61 therefore may no longer come together sufficiently to close the annulus and prevent blood flow back into the atrium 2. If left untreated, the FMR experienced in the state shown in FIG. 3A may overload the heart 1 and can possibly lead to or accelerate heart failure. Solutions presented herein provide devices and methods for moving the papillary muscles 15 closer to their previous position, which may advantageously reduce the occurrence of mitral regurgitation.

As shown in FIG. 3A, the leaflets 61 of the mitral valve 6 (or tricuspid valve) are not in a state of coaptation, resulting in an opening between the mitral valve leaflets 61 during the systolic phase of the cardiac cycle, which allows the leakage flow 20 of fluid back up into the atrium 2. The papillary muscles 15 may be displaced due to dilation of the left ventricle 3, or due to one or more other conditions, as described above, which may contribute to the failure of the valve 6 to close properly. The failure of the valve leaflets 61 to coapt properly may result in unwanted flow in the outflow direction (e.g., the upward direction in FIG. 3A) and/or unwanted backflow or regurgitation toward the inflow direction (e.g., the downward direction in FIG. 2A).

FIG. 3B provides an overhead view of a mitral valve 6 (e.g., as viewed from the left atrium) in which the heart and/or left ventricle is in a state in which FMR is present. Relative positions of the papillary muscles are shown with respect to the mitral valve 6. As shown in FIG. 3B, dilation of the heart may result in displacement of the papillary muscles. For example, during normal conditions (e.g., when FMR is not present), a first papillary muscle 322 may be at a first position 322a and/or a second papillary muscle 323 may be at a first position 323a directly below and/or nearly directly below the coaptation line 21 of the mitral valve 6. As the heart dilates, one or papillary muscles may move further from being below the coaptation line 21 and/or a central portion of the mitral valve 6 and/or may move further from other papillary muscles. For example, the first papillary muscle 322 may migrate to a second position 322b and/or the second papillary muscle 323 may migrate to a second position 323b. As the papillary muscles migrate, chordae tendineae connected between the papillary muscles and the mitral valve 6 may cause force on the mitral valve 6. Accordingly, as the papillary muscles move further from the central portion of the mitral valve 6 and/or a coaptation line 21 of the mitral valve 6, the chordae tendineae may cause the leaflets to separate and/or the coaptation line 21 to open. For example, one or more leaflets 324 may be pulled from a first position 324a to a second position 324b, which may create greater separation between multiple leaflets.

Some embodiments disclosed herein provide solutions for treating HFrEF, HFpEF, and/or FMR using minimally invasive procedures and/or without the need for surgical procedures or destroying cardiac tissue. In particular, minimally invasive and/or passive techniques to improve valve performance are disclosed for improving cardiac function. Further, various embodiments disclosed herein provide for the treatment of HFrEF, HFpEF, and/or FMR that can be executed on a beating heart, thereby allowing for the ability to assess the efficacy of the treatment and potentially implement modification thereto without the need for bypass support.

Some embodiments involve remodeling one or more ventricles (e.g., reducing ventricular volume and/or restoring one or more papillary muscles) to restore valve function and/or improve ejection fraction. Ventricular remodeling (e.g., restoring one or more displaced papillary muscles) can potentially treat FMR and/or HFrEF by, for example, improving coaptation of valve leaflets attached to the papillary muscles by chordae tendineae. Some embodiments described herein involve papillary muscle approximation and/or restoration by delivering a suture assembly and/or one or more means for anchoring to the heart via a subclavian, transapical, transseptal, transfemoral, or other minimally invasive and/or percutaneous approach. The term “suture” is used herein according to its plain and ordinary meaning and may refer to any generally thin and/or elongate device configured for delivery into a human heart and/or for creating tension. A “suture” may include any line, string, cord, wire, and/or other similar device. A suture assembly may comprise one or more sutures or “branches.” The term “branch” is used herein according to its plain and ordinary meaning and may refer to a suture and/or portion of a suture. For example, a suture assembly may comprise a single suture and different portions of the single suture may be identified as separate “branches.” Moreover, a suture assembly may comprise two or more sutures and the two or more sutures may be identifiable as a single branch, two branches, or more than two branches. In some cases, a multiple “branches” of a suture assembly may be separate portions of the suture assembly extending outwardly in different direction from a common joint or from separate base portions.

The means for anchoring may include various anchoring elements which may be composed of metal (e.g., Nitinol or other shape-memory alloy, cobalt-chromium, and/or stainless steel), plastic, and/or a combination of materials. The anchoring elements may be configured to anchor to one or more areas of tissue by penetrating, contacting, pressing against, or otherwise engaging portions of tissue of a heart. An anchoring means may be configured to attach to a suture and/or a suture may be configured to be connected between multiple anchoring means. Sutures may be configured to apply pressure to and/or otherwise move the anchoring elements to a desired orientation and/or to cause a desired level of remodeling.

The means for anchoring may be positioned at various points in and/or around the heart using various delivery procedures. In a minimally invasive procedure, the apex or other region of the heart may be made accessible through an incision at or near the ribs. The means for anchoring and/or suture(s) may be placed along or within various ventricle walls and/or papillary muscles. As used herein, the term “ventricle wall” is used according to its broad and ordinary meaning and may refer to any area of tissue separating a ventricle of the heart from another chamber of the heart or an area outside the heart and may include, for example, the septum, posterior walls, and the region of the ventricle near the apex of the heart, among others. In some embodiments, different means for anchoring may be positioned at or near different ventricle walls. For example, a first anchoring element may be positioned within a ventricle and against an exterior of the septum and/or at least partially within the septum. A second anchoring element may be positioned at a first papillary muscle and/or at a second papillary muscle.

In some embodiments, a suture assembly and/or one or more anchoring elements for treating FMR, HFrEF, and/or other diseases may be delivered to an affected area of tissue via a minimally-invasive and/or transcatheter/percutaneous procedure. Various components of the suture assembly and/or anchoring elements may be delivered and adjusted using a transfemoral (artery), transapical, or transseptal procedure. Once in place, components of the suture assembly and/or anchoring elements may be detached from the delivery system and left in the heart as implants.

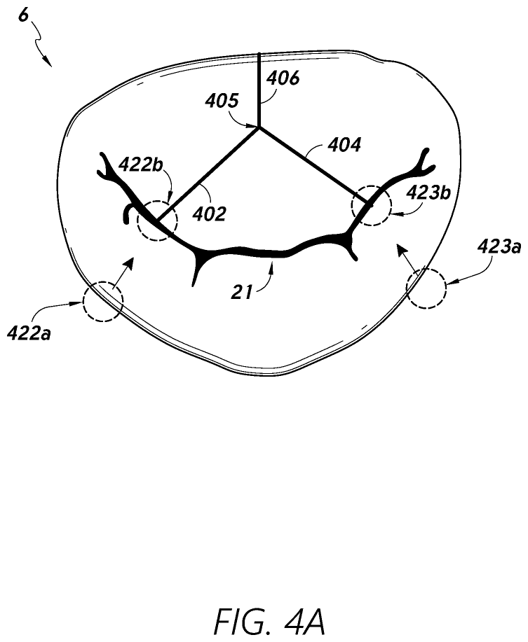

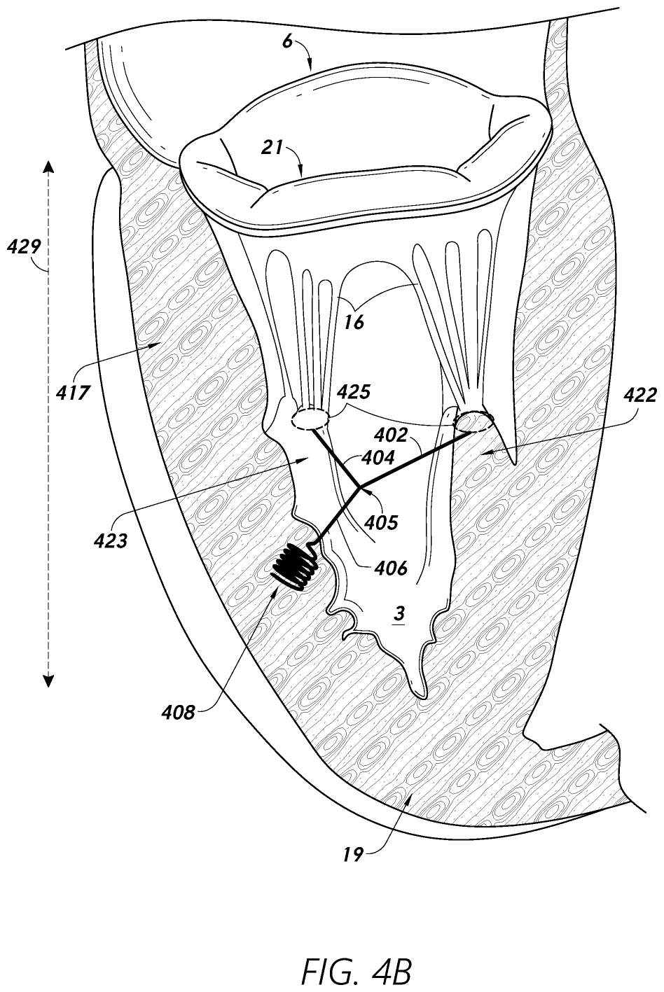

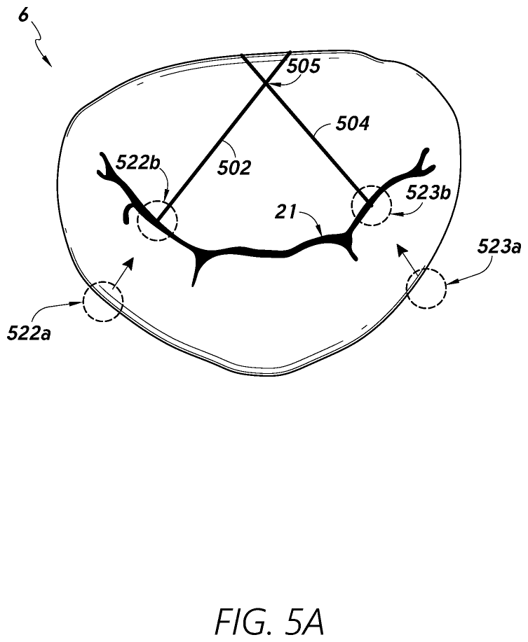

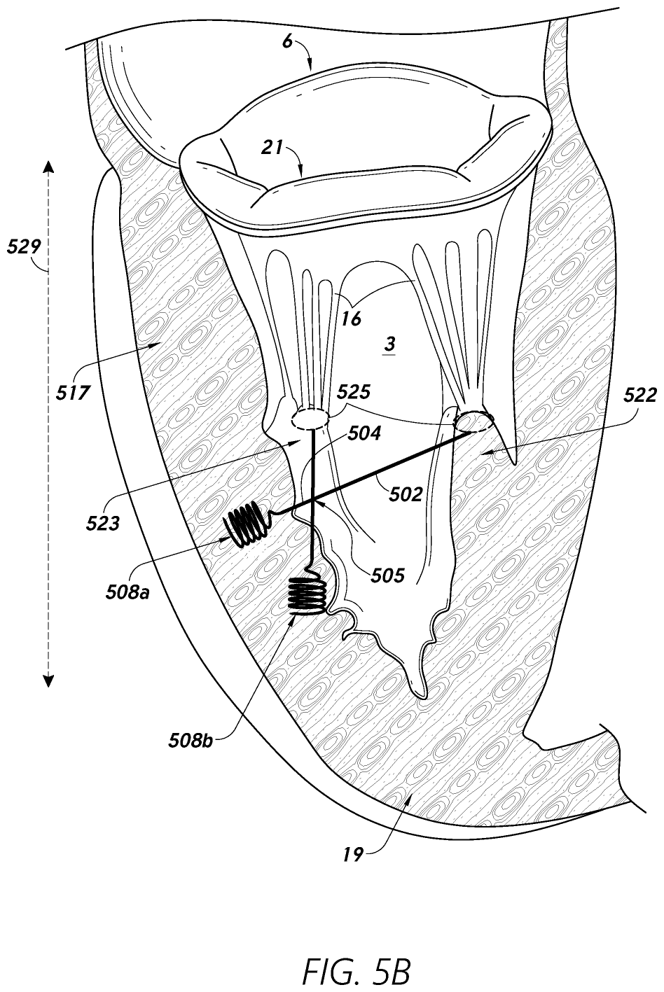

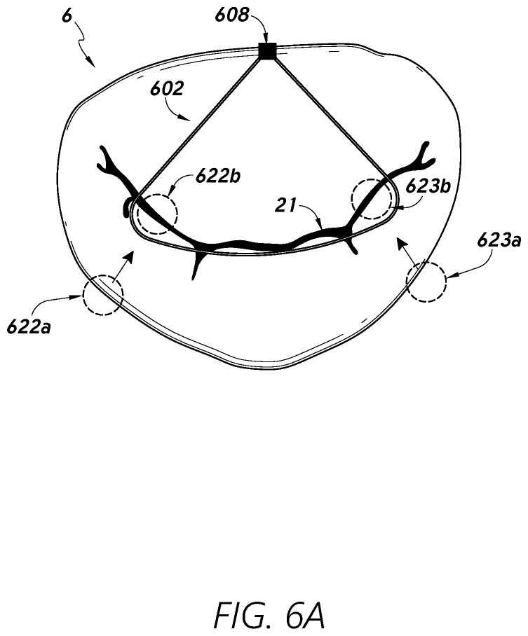

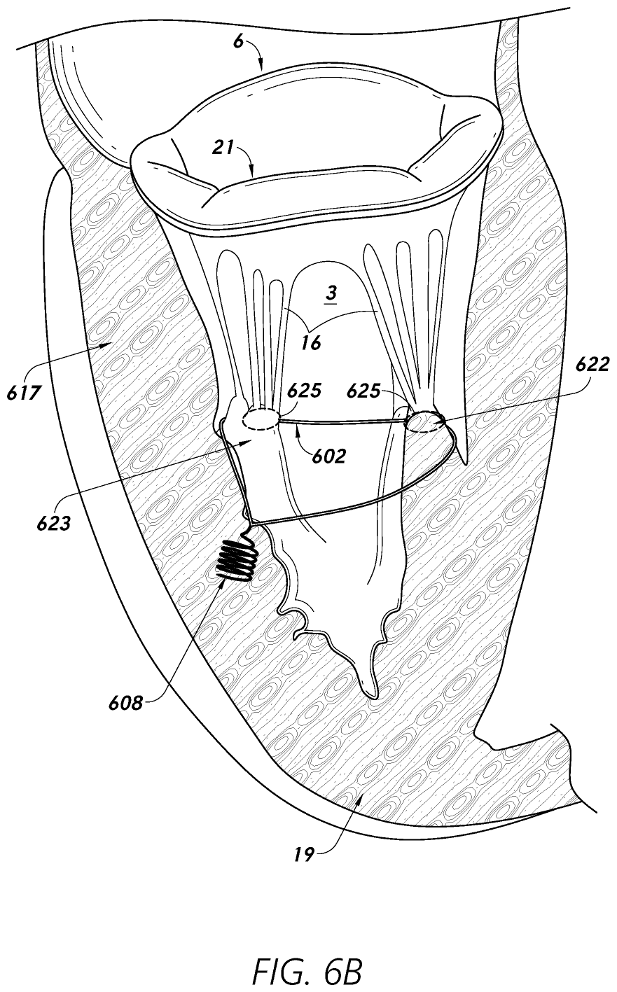

FIG. 4A provides an overhead view of the mitral valve 6 showing respective positions of portions of the papillary muscles and a restoration device comprising a suture assembly and one or more anchoring elements for correcting FMR and/or papillary muscle displacement. For illustrative purposes, FIG. 4A (and FIGS. 5A and 6A) provides the respective positions of at least portions of the papillary muscles using dashed circles. A first papillary muscle is represented in two alternate positions: a dilated and/or pre-restoration position 422a and a target/restored position 422b. Similarly, a second papillary muscle 423 is also represented in two alternate positions: a dilated and/or pre-restoration position 423a and a target/restored position 423b. The papillary muscles may not be visible from above the mitral valve, particularly when the coaptation line 21 of the mitral valve 6 is closed. However, the papillary muscles are shown here for illustrative purposes. Moreover, a papillary muscle may have varying widths and/or radii along a length of the papillary muscle and may not have the size/shape shown in FIG. 4A along the entire length of the papillary muscle.

In some embodiments, a restoration device may comprise a suture assembly and/or network comprising one or more sutures. The one or more sutures may extend in various directions to form one or more “branches.” The term “branch” is used herein in accordance with its plain and ordinary meaning and may refer to any portion, segment, and/or component of a suture assembly comprising one or more sutures. For example, branches may include portions of a suture extending in different directions from a common base portion and/or from a common joint. As shown in FIG. 4A, the suture assembly may comprise a first branch 402, second branch 404, and a third branch 406 extending from a joint 405. The first branch 402, second branch 404, and/or third branch may comprise a single suture, two sutures, three sutures, or more sutures. The joint 405 may represent a bend and/or change of direction of a single suture and/or of more than one sutures (e.g., for cases in which the suture assembly comprises a single suture or two sutures) and/or the joint 405 may represent a joining point between multiple sutures (e.g., for cases in which the suture assembly comprises more than one suture).

One or more sutures may be anchored to, connected to and/or otherwise engaged with the first papillary muscle 422. For example, the first suture/branch 402 may be anchored to the first papillary muscle 422 through use of an anchoring element and/or by wrapping the first suture/branch 402 around the first papillary muscle 422. An anchoring element may comprise a corkscrew or similar mechanism. Moreover, the first suture/branch 402 and/or a second suture/branch 404 may be anchored to and/or otherwise engaged with the second papillary muscle 423. While the suture assembly is shown in FIGS. 4A and 4B only being anchored to two papillary muscles, the suture assembly may be anchored to a single papillary muscle or three papillary muscles. The suture assembly may further be anchored to a ventricle wall such as the septum. For example, a third suture/branch 406 may be configured to be anchored to a ventricle wall. Moreover, in some embodiments, the suture assembly may not be anchored to any papillary muscles and/or may be anchored to multiple ventricle walls and/or two or more portions of a ventricle wall.

In some embodiments, the first suture/branch 402, second suture/branch 404, and or third suture/branch 406 may be interconnected. For example, the first suture/branch 402, second suture/branch 404, and/or third suture/branch 406 may be joined to each other at the joint 405 between the papillary muscles and/or the ventricle wall. In some embodiments, a suture/branch may be anchored to both the first papillary muscle 422 and the second papillary muscle 423. Another suture/branch may be anchored to the ventricle wall. The first suture/branch 402 and the second suture/branch 404 may be joined at the joint 405 between the papillary muscles and/or between a papillary muscle and the ventricle wall. For example, the first suture/branch 402 and/or the second suture/branch 404 may comprise one or more attachment mechanisms configured to attach to one or more papillary muscles, ventricle walls, and/or sutures. For example, the second suture/branch 404 may comprise a hook, loop, and/or other device configured to join with the first suture/branch 402 and/or a corresponding attachment mechanism at the first suture/branch 402.

In some embodiments, the first suture/branch 402, second suture/branch 404, and/or third suture/branch 406 may be joined prior to delivery into the heart. However, one or more sutures/branches may be joined after delivery to the heart. For example, a first suture may be delivered to the heart and may be anchored to the first papillary muscle 422 and/or the second papillary muscle 423. In some embodiments, after the first suture/branch 402 is anchored to the first papillary muscle 422 and/or the second papillary muscle 423, the second suture/branch 404 may be joined to the first suture/branch 402. The second suture/branch 404 may be anchored to the ventricle wall such that when the second suture is joined to the first suture/branch 402, the second suture/branch 404 may be configured to pull the first suture/branch 402 towards the ventricle wall and or the first suture/branch 402 may pull the second suture/branch 402 toward the papillary muscles. In some embodiments, one or more sutures/branches may be cinched to further apply pulling force to other sutures, papillary muscles, and/or ventricle walls. For example, a surgeon may pull or otherwise engage the second suture/branch 404 to pull the second suture/branch 404 closer to the ventricle wall, thereby pulling the first suture/branch 402 and/or one or more papillary muscles nearer to the ventricle wall.

As shown in FIG. 4A, force applied to the papillary muscles by the suture assembly may cause the papillary muscles to move nearer to positions that are directly below a central portion of the mitral valve and/or a coaptation line 21 of the mitral valve 6. Moreover, the force applied to the papillary muscles may cause the papillary muscles to move nearer to each other. In some embodiments, one or more sutures anchored to the papillary muscles may be configured to pull the papillary muscles directly and or nearly directly towards each other. As shown in FIG. 4A, a suture (e.g., the third suture/branch 406) anchored to the ventricle wall may be configured to cause the papillary muscles to move towards each other in an indirect manner. For example, the papillary muscles may be caused to move in a direct line towards an anchoring point at the ventricle wall. As the papillary muscles move towards the ventricle wall, the papillary muscles may move closer to each other.

Because the suture assembly and/or anchoring elements is/are configured for percutaneous delivery, such devices may provide for simplified delivery and a very low risk of complications associated with delivery. The suture assembly and/or anchoring elements may be delivered via a transcatheter procedure without requiring open-heart surgery.

FIG. 4B provides a side cross-sectional view of the left ventricle 3 with an implanted papillary muscle restoration device comprising a suture assembly and one or more anchoring elements 408. As shown in FIG. 4B, a suture (e.g. the third suture/branch 406) may be anchored at a ventricle wall 417 (e.g. the septum) through use of an anchoring element 408 which may include a corkscrew or similar device. Each of the papillary muscles (e.g., the first papillary muscle 422 and the second papillary muscle 423) may be connected to the mitral valve 6 or other valve by chordae tendineae 16. While the anchoring element 408 is shown embedded in the ventricle wall 417, the anchoring element 408 may be situated at least partially within the left ventricle 3, right ventricle, and/or other chamber of the heart. For example, one or more sutures and/or the anchoring element 408 may be delivered percutaneously to the right ventricle and/or may be passed through the septum into the left ventricle 3. In some embodiments, one or more sutures may be passed through the septum into the left ventricle 3 while the anchoring element 408 may be configured to remain situated in the right ventricle against the septum. If the one or more sutures and/or the anchoring element 408 are delivered percutaneously to the left ventricle 3, the anchoring element 408 may be anchored to an external portion of the ventricle wall and/or may be configured to be embedded within the ventricle wall 417. In some embodiments, the anchoring element 408 may comprise a corkscrew configured to be twisted and/or otherwise pressed into the ventricle wall 417 to provide a secure anchor for the one or more sutures.

The one or more sutures and/or the anchoring element 408 may be positioned at any point along the papillary muscle and/or ventricle wall 417. For example, the anchoring element 408 may be situated approximately in line along a vertical axis 429 with tips 425 of the papillary muscles (i.e., the portions of the papillary muscles that are attached to the chordae tendineae 16). However, the anchoring element 408 may be situated above or below the tips 425 of the papillary muscles along the vertical axis 429. In some cases, it may be advantageous to situate the anchoring element 408 generally close to an apex 19 portion of the heart. Similarly, one or more sutures may be anchored to any portion of the papillary muscles. In some cases, it may be advantageous to situate and/or anchor the one or more sutures near a tip 425 of the papillary muscle to maximize migration of at least the tip 425 of the papillary muscle. However, due to concerns of damaging the chordae tendineae 16 attached to the papillary muscles, the one or more sutures may be anchored a sufficient distance from the tips 425 of the papillary muscles. For example, during the restoration process of the papillary muscles, the one or more sutures may slide along and or otherwise move with respect to the papillary muscles. To prevent damage to the chordae tendineae 16, the one or more sutures may be anchored at the papillary muscles with some separation from the chordae tendineae 16.

In some embodiments, the one or more sutures may form a joint 405, which may represent a joining point of multiple sutures/branches and/or a bending point of one or more sutures/branches. The position of the joint 405 may be adjustable based on amount of cinching of any of the one or more sutures. For example, as one or more sutures are cinched, the joint 405 may move closer towards the ventricle wall 417 as the one or more sutures are configured to apply pulling force to the one or more papillary muscles.

In some embodiments, a suture assembly may not be employed in combination with one or more anchoring elements 408, as shown in FIG. 4B. For example, the third branch 406 of the suture assembly may be configured to extend through the ventricle wall 417 and out of the body. A surgeon may be able to pull on the third branch 406 to cause movement and/or migration of the papillary muscles 422, 423.

FIG. 5A provides an overhead view of mitral valve 6 showing relative positions of the papillary muscles and various components of a restoration device implanted within the left ventricle (e.g., below the mitral valve 6). In some embodiments, the restoration device may comprise a suture assembly comprising one or more sutures 502, 504 configured to apply pulling force to one or more papillary muscles 522, 523 to cause movement of the one or papillary muscles, 522, 523 towards a ventricle wall. The suture assembly may comprise multiple sutures and/or branches. For example, a first suture/branch 502 may be configured to be anchored to a first papillary muscle 522 and/or the ventricle wall.

A second suture/branch 504 may be configured to be anchored to a second papillary muscle 523 and/or a ventricle wall. The first suture/branch 502 and/or the second suture/branch 504 may be anchored to a common ventricle wall and/or to different ventricle walls. In some embodiments, multiple sutures/branches may be configured to contact each other and/or at least partially overlap. For example, as shown in FIG. 5A, the first suture/branch 502 may be configured to be anchored to a first papillary muscle 522 and may be configured to be anchored to the ventricle wall at a point of ventricle wall that is nearer the second papillary muscle 523 than the first papillary muscle 522 and/or nearer the second papillary muscle 523 than an anchoring point of the second suture/branch 504 at the ventricle wall. In some embodiments, the first suture/branch 502 may not be used in combination with the second suture/branch 504. For example, only the first suture/branch 502 or only the second suture/branch 504 may be used to restore the first papillary muscle 522 and/or the second papillary muscle 523.

The second suture/branch 504 may be configured to be anchored to the second papillary muscle 523 and to a point of the ventricle wall that is nearer the first papillary muscle 522 than the second papillary muscle 523 and/or nearer the second papillary muscle 523 than an anchoring point of the first suture/branch 502 at the ventricle wall. Accordingly, the first suture/branch 502 and the second suture/branch 504 may cross paths and/or may overlap to some extent (e.g., as viewed from above) at an intersection point 505. In some embodiments, the first suture/branch 502 and the second suture/branch 504 may be anchored to the ventricle wall and/or the papillary muscles 522, 523 at generally equivalent positions along a vertical axis 529 (i.e., into the page of FIG. 5A). Accordingly, as the first suture/branch 502 and second suture/branch 504 cross paths and/or at least partially overlap, the first suture/branch 502 and the second suture/branch 504 may contact each other and/or may be joined with each other. However, the first suture/branch 502 and second suture/branch 504 may be configured to be anchored at any points along the ventricle wall and/or papillary muscles 522, 523 along the vertical axis 529. Accordingly, as the first suture/branch 502 and second suture/branch 504 cross paths and/or at least partially overlap, the first suture/branch 502 and second suture/branch 504 may not contact each other.

The one or more sutures may be configured to be anchored to papillary muscles 522, 523 that may be at least partially displaced due to dilation of the heart. A first position 522a of the first papillary muscle 522 may represent a displaced position of the first papillary muscle and a first position 523a of the second papillary muscle 523 may represent a displaced position of the second papillary muscle 523. Accordingly, the one or more sutures/branches may be configured to extend from the ventricle wall 517 to the displaced positions of the papillary muscles.

A second position 522b of the first papillary muscle 522 may represent a target/desired position of the first papillary muscle 522 and a second position 523b may represent a target/desired position of the second papillary muscle 523. The one or more sutures/branches 502, 504 may be configured to be cinched to apply pulling force to the one or papillary muscles 522, 523 to move the one or more papillary muscles 522, 523 towards the ventricle wall 517. In some embodiments, the one or more sutures/branches 502, 504 may be configured to be individually cinched and/or may be configured to be cinched jointly. For example, the one or more sutures/branches 502, 504 may be joined at the intersection point 505 and/or at another intersection point at the ventricle wall 517 and/or another point. Accordingly, a surgeon may pull on and or otherwise engage a single suture/branch and/or device to cause cinching of each of the sutures/branches 502, 504.

Each of the sutures/branches 502, 504 may be configured to cause migration of at least one of the papillary muscles towards an anchoring point of the suture. In some embodiments, each of the sutures/branches 502, 504 may be configured to be anchored to different points of the ventricle wall 517 and/or to different ventricle walls. Accordingly, each suture/branch 502, 504 may be configured to cause movement of a papillary muscle 522, 523 to a different point of the ventricle wall 517 and/or to different ventricle walls.

Due to the overlapping positioning of the one or more sutures/branches 502, 504 as shown in FIGS. 5A and 5B, cinching of the one or more sutures/branches 502, 504 may be configured to cause movement of the papillary muscles 522, 523 towards the ventricle wall 517 and/or closer to one another. Moreover, cinching of the sutures/branches 502, 504 may be configured to cause movement of the papillary muscles 522, 523 closer to being directly below the coaptation line 21 of the mitral valve 6 (e.g., as viewed from the left atrium).

FIG. 5B provides a cross/sectional side view of a left ventricle 3 with an implanted restoration device comprising a suture assembly and one or more anchoring elements 508a, 508b. As shown in FIG. 5B, a first suture/branch 502 may be configured to be anchored to a ventricle wall 517 (e.g., the septum) via a first anchoring element 508a and a second suture/branch 504 may be configured to be anchored to the ventricle wall 517 via a second anchoring element 508b.

While the anchoring elements 508a, 508b are shown in FIG. 5B being at different positions along the vertical axis 529 of the ventricle wall 517, the first anchoring element 508a, second anchoring element 508b, and/or additional anchoring elements may be situated along a common plane along the vertical axis 529. In some cases, it may be advantageous to situate at least one of the one or more anchoring elements between a tip 525 portion of a papillary muscle and an apex 19 region of the heart. Similarly, the one or more sutures/branches may be configured to be anchored to and/or otherwise engaged with the one or more papillary muscles 522, 523 at any point along the one or papillary muscles.

In some embodiments, the one or more sutures/branches 502, 504 may be configured to penetrate the surface of the one or more papillary muscles 522, 523. However, the one or more sutures/branches 502, 504 may be configured to wrap around and/or be anchored to an exterior surface of the one or more papillary muscles 522, 523 to cause minimal damage to the papillary muscle. While FIG. 5B shows the anchoring elements 508a, 508b embedded in the ventricle wall 517, one or more of anchoring elements 508a, 508b may be configured to be anchored to an external surface of the ventricle wall 517. For example, an anchoring element 508a, 508b may be situated on the right ventricle side of the septum and/or a suture (e.g., the first suture/branch 502) may pass through the septum to be anchored to papillary muscles 522, 523 in the left ventricle 3. In some cases, dilation of the ventricle may cause the papillary muscles to move away from the ventricle wall 517. By anchoring the sutures/branches 502, 504 to the papillary muscles 522, 523, the sutures/branches 502, 504 may be configured to apply pulling force to one or more papillary muscles 522, 523 to cause movement of the papillary muscles towards the ventricle wall 517.

FIG. 6A provides an overhead view of the mitral valve 6 (e.g., as viewed from the left atrium) showing respective positions of at least tip portions of the papillary muscles and one or more components of a restoration device implanted in the left ventricle 3. In some embodiments, one or more sutures 602 may be configured to wrap around, penetrate, and/or otherwise engage one or more papillary muscles (e.g., a first papillary muscle 622 and/or a second papillary muscle 623).

As shown in FIG. 6A, one or more sutures 602 may be anchored to a ventricle wall through use of one or more anchoring elements 608. The one or more anchoring elements 608 may be configured to anchor the one or more sutures 602 to a ventricle wall (e.g., the septum). In some embodiments, the anchoring element 608 may be configured to be situated in the same anatomical chamber (e.g., the left ventricle 3) as the one or more sutures 602. For example, an anchoring element 608 may configured to be situated at an exterior portion of a ventricle wall while the one or more sutures 602 may be configured to be situated at least partially within the ventricle (e.g., the left ventricle 3).

Additionally or alternatively, an anchoring element 608 may configured to be situated and/or embedded within a ventricle wall and/or situated in a different anatomical chamber (e.g., the right ventricle) than the one or more sutures 602. For example, an anchoring element 608 may be situated against a right ventricle side of the septum while the one or more sutures 602 may be configured to pass through the septum and into the left ventricle. While FIGS. 6A and 6B show two papillary muscles, in some embodiments, one or more sutures 602 may be configured for restoring and/or reshaping a single papillary muscle and/or three papillary muscles.

In some embodiments, one or more free ends of one or more sutures may be configured to extend from the anchoring element 608, wrap around one or more papillary muscles, and/or attach to the anchoring element 608. As shown in FIG. 6A, a single suture 602 and/or two or more joined sutures may be configured to extend from the anchoring element 608 and wrap around two papillary muscles 622, 623 and form a closed loop at the anchoring element 608. Accordingly, the one or more sutures 602 may be configured to form a triangular shape having ends at the anchoring element 608, a first papillary muscle 622, and a second papillary muscle 623.

The one or more sutures 602 may be configured to wrap around the one or papillary muscles when the papillary muscles are in a dilated state and/or at dilated positions 622a, 623a. The one or more sutures 602 may be configured to be cinched to cause the papillary muscles to move towards target/restored positions 622b, 623b (i.e., the first papillary muscle 622 may move from a dilated position 622a to a target/restored position 622b).

In some embodiments, cinching the one or more suture 602 may be configured to cause movement of the papillary muscles 622, 623 closer to the ventricle wall and/or closer to each other. Cinching the one or more sutures may cause the one or more sutures 602 to form a triangular shape having a smaller surface area than at first delivery of the one or more sutures 602. Following cinching of the one or more sutures 602, the papillary muscles 622, 623 may be situated directly or nearly directly below the coaptation line 21 of the mitral valve 6.

FIG. 6B provides a cross-sectional side view of the left ventricle 3 with an implanted restoration device comprising a suture assembly and an anchoring element 608. The suture assembly may comprise one or more sutures 602. As shown in FIG. 6B, multiple ends of the suture assembly may be anchored to and/or attached to an anchoring element 608. The anchoring element 608 may be at least partially embedded in the ventricle wall 617 (e.g., the septum 17) and/or anchored to an external surface of the ventricle wall 617.

The anchoring element 608 may be situated on the left ventricle 3 or right ventricle side of the wall and/or at an external surface of the heart. In some embodiments, the one or more sutures 602 may be configured to wrap around one or more papillary muscles 622, 623 at any point between a tip 625 portion and a base portion of the papillary muscles. In some embodiments, the one or more sutures 602 may be configured to be wrapped around and/or otherwise anchored to a portion of the papillary muscle that is sufficiently distal from the chordae tendineae 16 to prevent damage to the chordae tendineae 16. The anchoring element 608 may be situated at any position within and/or at the ventricle wall 617. In some embodiments, the anchoring element 608 may be situated at or near an apex 19 region of the heart and/or between the apex 19 and the tip 625 portions of the papillary muscles 622, 623.

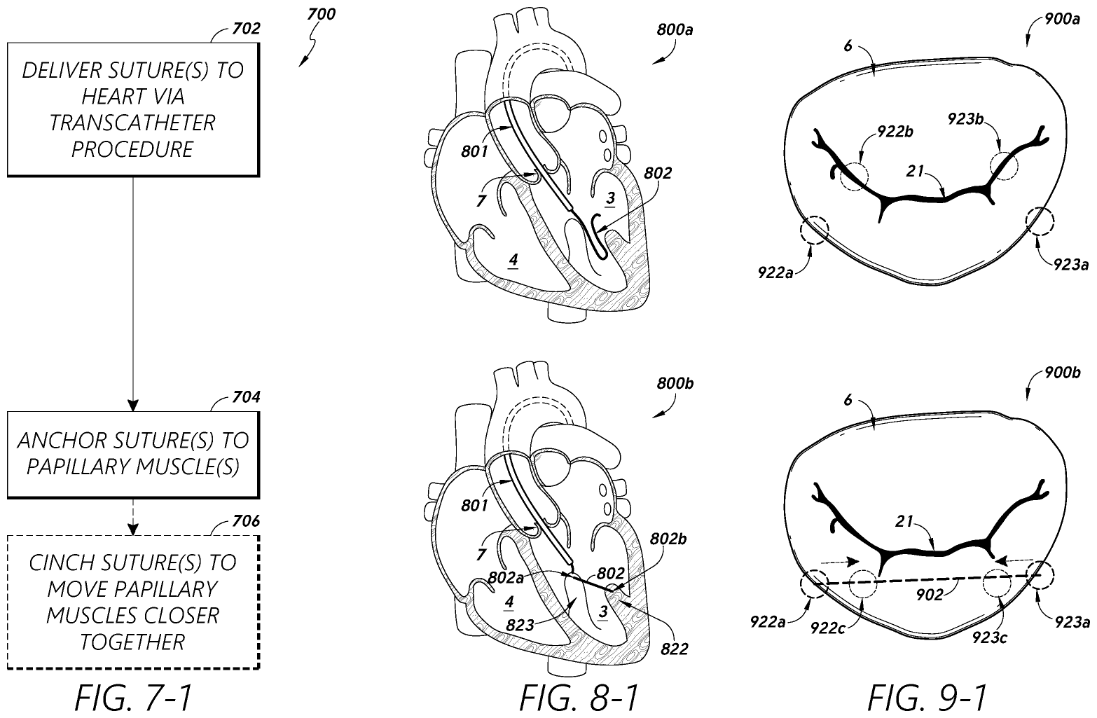

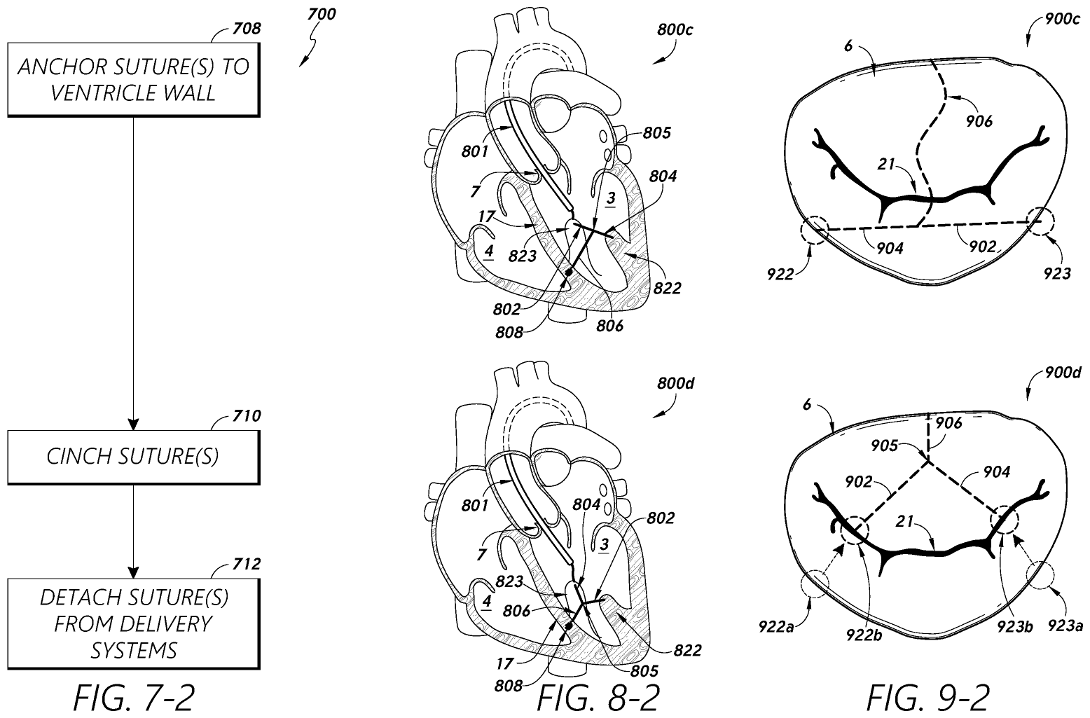

FIG. 7 provides a flow chart for a transcatheter process for delivering a restoration device to a heart. FIG. 8 provides cross-sectional views of the heart for various steps of the transfemoral process of FIG. 7. FIG. 9 provides overhead views of the mitral valve 6 including respective positions of the papillary muscles and restoration device components for various stages of the flow chart represented in FIG. 7. While some steps of the process 700 may be directed to the left ventricle, such steps may also be applied to the right ventricle.

At block 702, the process 700 involves delivering one or more sutures to the heart percutaneously and/or via a transcatheter procedure. As shown in image 800a of FIG. 8, a catheter 801 may be delivered via the aortic valve 7 into the left ventricle 3. Additionally or alternatively, one or more catheters 801 may be delivered into the right ventricle 4. The catheter 801 may be configured for delivery of one or more sutures 802 and/or anchoring elements through and/or into the heart.

As shown in image 900a, the heart may be in a dilated state at delivery of the one or more sutures. In some cases, one or more papillary muscles 822, 823 may be displaced due to dilation of the heart. Displacement of the papillary muscles 822, 823 may cause the one or more papillary muscles 822, 823 to move away from a central portion and/or coaptation line 21 of the mitral valve. As shown in image 900a, at least a portion of a first papillary muscle 922 may be situated at a dilated position 922a and/or may be otherwise displaced from a normal/restored position 922b. Similarly, at least a portion of a second papillary muscle 923 may be situated at a dilated position 923a and/or may be otherwise displaced from a normal/restored position 923b.

The one or more sutures (i.e., suture assembly) and/or anchoring elements may be delivered using a transcatheter procedure. For example, the suture assembly and/or anchoring elements may be delivered using a transfemoral, transendocardial, transcoronary, transseptal, and/or transapical procedure, or other approach. One or more sutures and/or one or more anchoring elements may be delivered via femoral vein and/or aortic valve 7 into the left ventricle 3. In some embodiments, one or more sutures and/or anchoring elements may be delivered via the superior vena cava, right atrium, and finally into the right ventricle 4. In optional embodiments, one or more sutures and/or anchoring elements may be introduced into a desired location via an open-chest surgical procedure, or using other surgical or non-surgical techniques known in the art.

In some embodiments, at least some components may be inserted into the right ventricle 4 (e.g., through the pulmonary valve or tricuspid valve) where the suture assembly may be configured to remodel the right ventricle or may be passed through the septum into the left ventricle. Alternatively, the suture assembly and/or one or more anchoring elements may be inserted into the left ventricle (e.g., through the aortic valve 7 or mitral valve 6) where the devices may be configured to remodel the left ventricle 3 or may be passed through the septum 17 into the right ventricle 4. For a transapical procedure, one or more sutures and/or anchoring elements may be inserted through the apex via a catheter. In optional embodiments, one or more sutures and/or anchoring elements may be delivered to a location outside of the heart for purposes other than restoring papillary muscles.

In some embodiments, one or more sutures and/or anchoring elements may be fed through a catheter 801 (e.g., a transfemoral catheter) that may be inserted into the left ventricle 3 and/or right ventricle 4. Needles and/or other devices may be passed through the catheter 801 to penetrate the septum 17 and/or other ventricle wall(s). For example, a transseptal needle may be introduced to pass through the septum 17 from the left ventricle 3 and may be embedded in the septum 17 and/or may pass into the right ventricle 4. The catheter 801 may be sized to accommodate the various sutures and/or anchoring elements. For example, the catheter may have a diameter of at least 12 French to fit anchoring elements having a diameter equal to or less than 12 French.

At block 704, the process 700 involves anchoring one or more sutures to one or more papillary muscles. In some embodiments, a single suture and/or two or more joint sutures may be anchored to a first papillary muscle and/or a second papillary muscle. As shown in image 800b, a first end 802a of a first suture/branch 802 may be anchored to a first papillary muscle 822 and/or a second end 802b of the first suture/branch 802 and/or a first end of a second suture/branch may be anchored to a second papillary muscle 823.

In some embodiments, one or more anchoring elements may be used to anchor the one or more sutures 802 to the papillary muscles 822, 823. For example, an anchoring element (e.g., a corkscrew anchor) may be embedded into a papillary muscle and the one or more sutures may be attached to the anchoring element. In some embodiments, one or more sutures may comprise an anchoring element. For example, a suture may extend into and/or may be joined to a corkscrew anchor, hook anchor, and/or another anchoring element.

In some cases, an anchoring element may be delivered to a papillary muscle prior to delivery of a suture. However, an anchoring element may additionally or alternatively be delivered in conjunction with one or more sutures, for example in situations when a suture is joined with the anchoring element. As shown in image 900a, one or more sutures 902 may be anchored to the one or more papillary muscles 922, 923 in the dilated state of the heart in which the papillary muscles 922, 923 are at dilated positions 922a, 923a and/or otherwise removed from target/restored positions 922b, 923b, which may be approximately below a central portion and/or coaptation line 21 of the mitral valve 6.

Block 706 provides an optional step of the process 700 in which one or more sutures anchored to the papillary muscles may be cinched to move the papillary muscles closer to each other. For example, a single suture (e.g., a first suture 802 in image 800b) and/or two or more joined sutures/branches of a suture assembly may be anchored between two or more papillary muscles (e.g., a first papillary muscle 822 and a second papillary muscle 823). Accordingly, as the suture and/or one or more sutures are cinched, the one or more sutures may cause pulling force on the papillary muscles to move the papillary muscles 922, 923 closer together (e.g., from dilated positions 922a, 923a to intermediate positions 922c, 923c) as shown in image 900b.

In some embodiments, cinching the one or more sutures 902 may cause movement of the papillary muscles 922, 923 in direct lines toward each other, as shown in image 900c. However, cinching of one or more sutures may additionally or alternatively cause the papillary muscles to indirectly move towards each other. For example, cinching of the sutures may cause each of the papillary muscles to move towards a common point of a ventricle wall and/or towards a position directly or nearly directly below a center portion and/or coaptation line of the mitral valve 6. By doing so, the papillary muscles may be moved closer together by virtue of moving towards a common point.

At block 708, the process 700 involves anchoring one or more sutures to a ventricle wall (e.g., the septum 17). As shown in image 800c, a second suture 804 of a suture assembly may be anchored to the septum 17 or another ventricle wall. One or more sutures anchored to the ventricle wall may be configured to be joined with one or more other branches of the suture assembly anchored to papillary muscles. In some embodiments, the suture assembly may comprise a single suture.

As shown in image 800c, the suture assembly may form a T-shape having at least three branches in which a first suture/branch 802 is anchored to a first papillary muscle 822, a second suture/branch 804 is anchored to a second papillary muscle 823, and a third suture/branch 806 is anchored to a ventricle wall (e.g., the septum 17). Each of the first suture/branch 802, second suture/branch 804, and third suture/branch 806 may be joined at a junction point 805. Prior to cinching of at least a portion of the suture assembly, the junction point 805 may be situated approximately between the first papillary muscle 822 and the second papillary muscle 823. The various sutures/branches may be anchored to the septum 17 and/or other ventricle wall via one or more anchoring elements 808.

In some embodiments, the one or more sutures may be anchored to the ventricle wall for use of an anchoring element such as a corkscrew, a screw, a nail, a hook, a barb, and/or other mechanism. Image 900c provides an overhead view of the mitral valve including respective positions of the papillary muscles 922, 923 and components of the restoration device. In some embodiments, a third suture/branch 906 of a suture assembly may have a slackened, loose, and/or otherwise un-cinched form, as shown in image 900c, when anchored to a ventricle wall. The third suture/branch 906 may be configured to be cinched by pulling on the third suture/branch 906. For example, an end of the third suture/branch 906 may be accessible to a surgeon outside the body.

At block 710, the process 700 involves cinching at least some portions of the suture assembly. For example, the third suture/branch 806 of the suture assembly may be cinched, as shown in image 800d of FIG. 8 and image 900d of FIG. 9. Cinching at least some portions of the suture assembly may be configured to cause restoration and/or movement of one or more papillary muscles.

In some embodiments, cinching the suture assembly may be performed manually. For example, a surgeon may manually pull on or otherwise engage a suture and/or device connected to a suture to cause the suture assembly to be cinched. In some embodiments, an anchoring element at a ventricle wall may be configured to facilitate cinching the one or more sutures. For example, the anchoring element may comprise a ratchet and/or similar mechanism configured to allow one-way cinching and/or movement of the suture through the anchoring element and/or to prevent loosening of the suture assembly.

In some embodiments, the anchoring element may be configured to perform automatic cinching of the one or more sutures. For example, the anchoring element may be configured to twist and/or otherwise engage the suture assembly to cause cinching of the suture assembly.

As shown in images 800d and 900d, cinching of the one or more sutures may cause movement of the joint 805, 905 between the one or more branches of the suture assembly. The joint 805, 905 may be moved closer to the ventricle wall (e.g., the septum 17) as a result of the cinching. Moreover, the one or more papillary muscles and/or the first suture/branch 802, 902 and/or second suture/branch 804, 904 may be moved nearer to the ventricle wall and/or nearer to each other as a result of the cinching of the suture assembly. After the suture assembly is cinched, the joint 805, 905 may be approximately equidistant from the first papillary muscle 822, 922, the second papillary muscle 823, 923, and the ventricle wall. Moreover, the joint 805, 905 may represent a middle portion and/or a center portion of a triangle formed by the first papillary muscle 822, 922, second papillary muscle 823, 923, and the anchoring point of the third suture/branch 806, 906 at the ventricle wall.

At block 712, the process 700 may involve detaching the suture assembly (e.g., the one or more sutures and/or anchoring elements) from the delivery systems, which may include catheters and or other delivery devices. In some embodiments, detaching the one or more sutures and/or one or more anchoring elements may involve cutting one or more sutures.

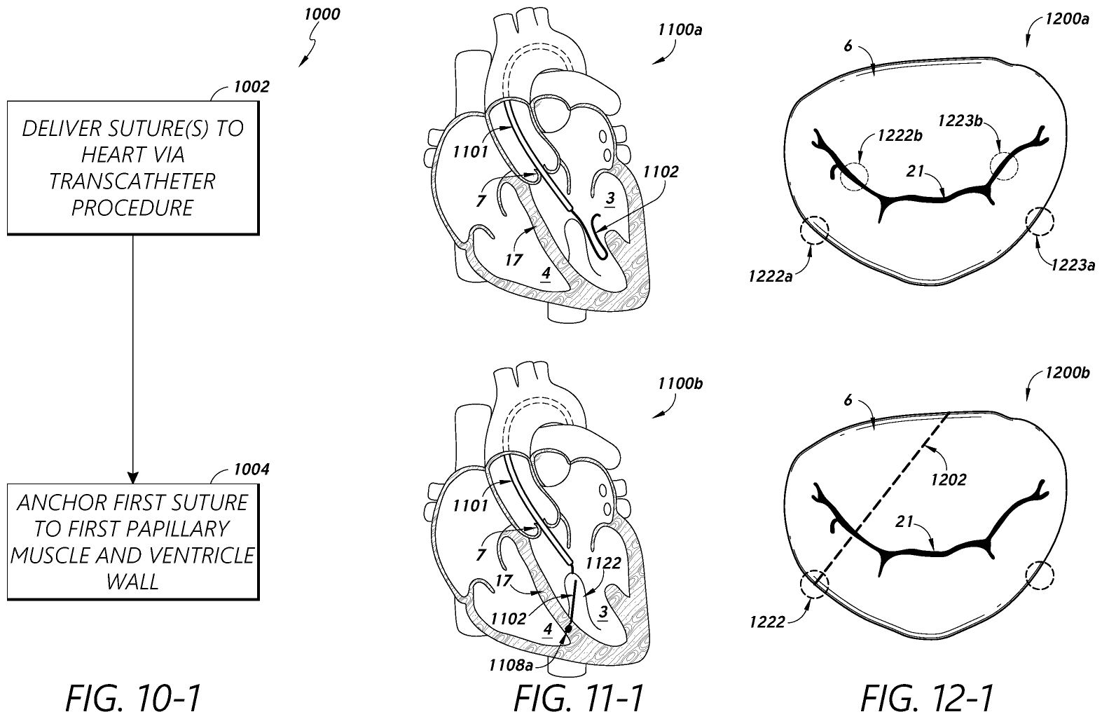

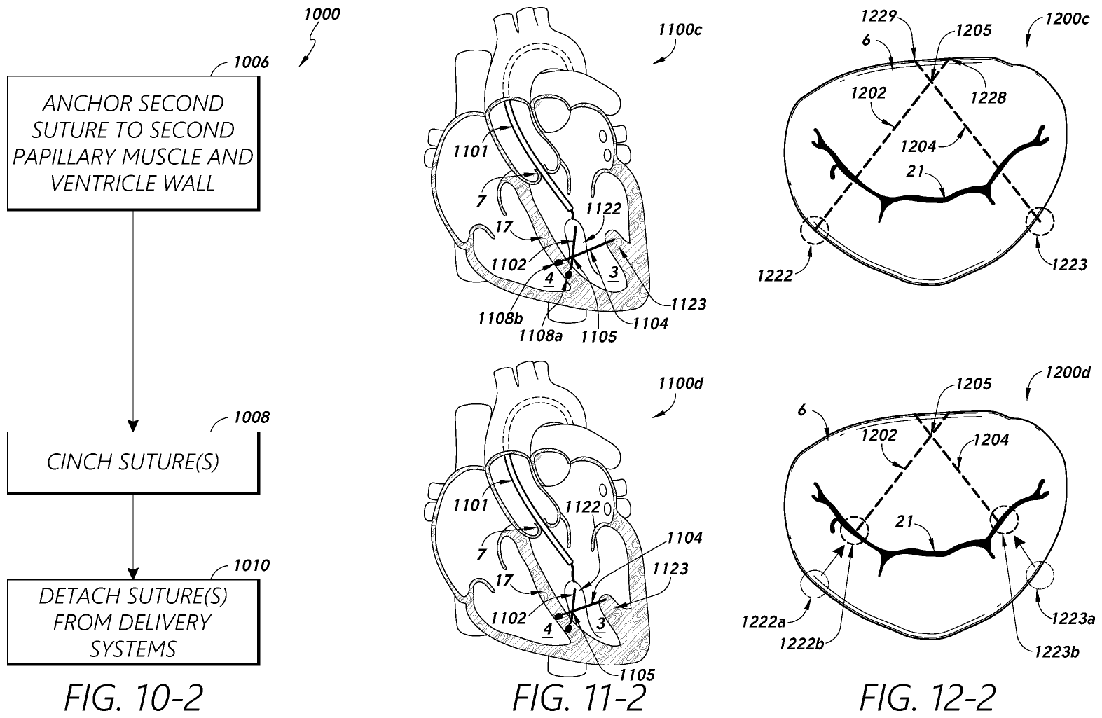

FIG. 10 provides a flowchart for a transcatheter process 1000 for delivering a shaping device to a heart and specifically to a chamber of the heart (e.g., the left ventricle). FIG. 11 provides cross-sectional side views of a heart corresponding to various steps of the process of FIG. 10. FIG. 12 provides overhead views of a mitral valve and respective positions of papillary muscles and restoration devices corresponding to various steps of the process 1000 of FIG. 10.

At block 1002, the process 1000 involves delivering one or more sutures to the heart via a transcatheter and/or transfemoral procedure. As shown in image 1100a of FIG. 11, one or more sutures, including a first suture 1102, may be delivered to the left ventricle 3 via the aortic valve 7 using a catheter 1101 and/or similar delivery device. While FIG. 11 shows images depicting a transfemoral process, the process 1000 described in FIG. 10 may be performed via other procedures, including delivery to the right ventricle.

As shown in image 1100a, a catheter 1101 and/or other delivery systems may be delivered through the pulmonary vein 9 into the left ventricle. Additionally and/or alternatively, a catheter 1101 and/or similar delivery systems may be delivered into the right ventricle and/or through the apex region 19 of the heart into the left ventricle 3.

As shown in image 1200a, the one or more sutures may be delivered to the heart when the heart is in a dilated state and/or when the papillary muscles 1222, 1223 are displaced from being directly and/or nearly directly below a central position and/or coaptation line 21 of the mitral valve 6. For example, the first papillary muscle 1222 and/or second papillary muscle 1223 may be positioned at dilated positions 1222a, 1223a, respectively, rather than at target/desired positions 1222b, 1223b, respectively.

At block 1004 and as shown in image 1100b of FIG. 11 and image 1200b of FIG. 12, the process 1000 involves anchoring a first suture/branch 1102, 1202 of the suture assembly to a first papillary muscle 1122, 1222 and to a ventricle wall (e.g., the septum 17). The first suture 1102, 1202 may have a slackened, loose, and/or otherwise un-cinched form when anchored to the first papillary muscle 1122, 1222 and/or to the ventricle wall. In some embodiments, the first suture 1102, 1202 may be anchored at a point of the ventricle wall such that the first suture 1102, 1202 may form a generally 45° angle (e.g., when viewed from above, as shown in image 1200b of FIG. 12) between a coaptation line 21 of the mitral valve 6 and the ventricle wall.

The first suture 1102, 1202 may be configured to penetrate, wrap around, and/or otherwise attach to the first papillary muscle 1122, 1222 using any suitable means. Similarly, the first suture 1102, 1202 may be configured to penetrate and/or attach to a first anchoring element 1108a positioned within the ventricle wall and/or at an external surface of the ventricle wall. The first suture 1102, 1202 may be anchored to the first papillary muscle 1122, 1222 and/or the ventricle wall in a slackened and or otherwise un-cinched state. In other words, the first suture 1102, 1202 may be configured to be cinched to apply pulling force of the first papillary muscle 1122, 1222.

At block 1006 and as shown in image 1100c of FIG. 11 and image 1200c of FIG. 12, the process 1000 involves anchoring a second suture/branch 1104, 1204 to a second papillary muscle 1123, 1223 and to a ventricle wall (e.g., the septum 17). The second suture 1104, 1204 may have a slackened, loose, and/or otherwise un-cinched form when anchored to the second papillary muscle 1123, 1223 and/or to the ventricle wall. In some embodiments, the second suture 1104, 1204 may be anchored at a point of the ventricle wall such that the second suture 1104, 1204 may be configured to form a generally 45° angle (when viewed from above, as shown in FIG. 12) between a coaptation line 21 of the mitral valve 6 and the ventricle wall.

The second suture 1104, 1204 may be configured to penetrate, wrap around, and/or attach to the second papillary muscle 1123, 1223 using any suitable means. Moreover, the second suture 1104, 1204 may be configured to attach, extend from, and/or anchor to a second anchoring element 1108b which may be anchored within and/or to the ventricle wall. Similarly, the first suture 1102, 1202 may be configured to penetrate and/or attach to a first anchoring element 1108a positioned within the ventricle wall and/or at an external surface of the ventricle wall. The first suture 1102, 1202 and/or second suture 1104, 1204 may be configured to be anchored to the second papillary muscle 1123, 1223 and/or the ventricle wall in a slackened and or otherwise un-cinched state. In other words, the second suture 1104, 1204 may be configured to be cinched to apply pulling force of the second papillary muscle 1123, 1223.

As shown in image 1200c, the second suture/branch 1204 may be anchored to the ventricle wall at a second anchoring point 1229 which is nearer to the first papillary muscle 1222 than a first anchoring point 1228 of the first suture 1202. Accordingly, the first suture 1202 and the second suture 1204 may cross paths and/or overlap. Alternatively, the first suture 1202 and second suture 1204 may not overlap and/or may be anchored to a common point at the ventricle wall.

In some embodiments, the second suture 1204 may be configured to anchor to a point of the ventricle wall that is nearer to the second papillary muscle 1223 than an anchoring point of the first suture 1202. Accordingly, there may be no overlap between the first suture 1202 and the second suture 1204. In some embodiments, there may be at least partial overlap between the first suture 1202 and the second suture 1204 such that the first suture 1202 and the second branch 1202 form an overlap point 1205 (see also the overlap point 1105 in image 1100c of FIG. 11). The overlap point 1105, 1205 may be configured to be situated approximately equidistant from the first papillary muscle 1222 and the second papillary muscle 1223.

Each of the first suture 1202 and the second suture 1204 may be configured to be anchored at a point and/or different points at the ventricle wall. As shown in image 1100c, the first suture 1202 and the second suture 1204 may have anchoring points that may be displaced from each other along a vertical axis (i.e., from the mitral valve 6 to the apex 19). In other words, the first suture 1202 may be anchored to a point of the ventricle wall and/or to a first anchoring element 1108a that may be situated nearer to the apex 19 region of the heart than an anchoring point of the second suture 1204 and/or a second anchoring element 1108b is to the apex 19 region.

At block 1008, the process 1000 involves cinching the one or more sutures of the suture assembly, as shown in image 1100d of FIG. 11 and image 1200d of FIG. 12. Cinching the first suture 1102, 1202 and/or the second suture 1104, 1204 may involve reducing the distance between a ventricle wall (e.g., the septum 17) and one or more ends of the suture assembly anchored to the papillary muscle(s). In this way, cinching of the one or more sutures may cause movement of the papillary muscles towards the ventricle wall.

In some cases, the papillary muscles may be configured to move towards each other as they move towards the ventricle wall. The first suture 1102, 1202 and/or the second suture 1104, 1204 may be cinched until a desired amount of restoration and/or reshaping is achieved. For example, as shown in image 1200d of FIG. 12, the first suture 1202 and/or the second suture 1204 may be cinched until the first papillary muscle 1222 moves from a dilated position 1222a to a target/restored position 1222b and/or the second papillary muscle 1223 approximately moves from a dilated position 1223a to a target/restored position 1223b and/or a central position beneath the mitral valve 6. In some embodiments, the first suture 1102, 1202 and/or the second suture 1102, 1202 may be cinched until the first papillary muscle 1122, 1222 and/or the second papillary muscle 1123, 1223 is directly and/or nearly directly beneath the coaptation line 21 of the mitral valve 6 when viewed from the left atrium as in FIG. 12.

At block 1010, the process 1000 involves detaching the one or more sutures from various delivery systems. For example, the first suture 1102, second suture 1104, first anchoring element 1108a, and/or second anchoring element 1108b shown in FIG. 11 may be detached from the catheter 1101 and/or other devices.

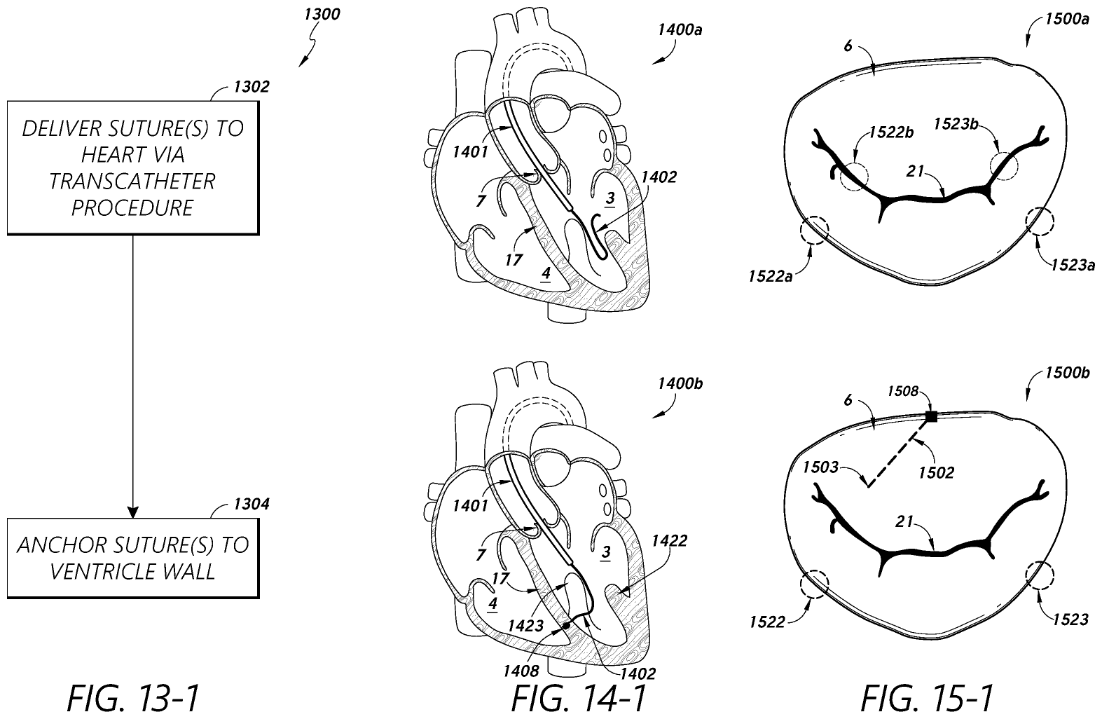

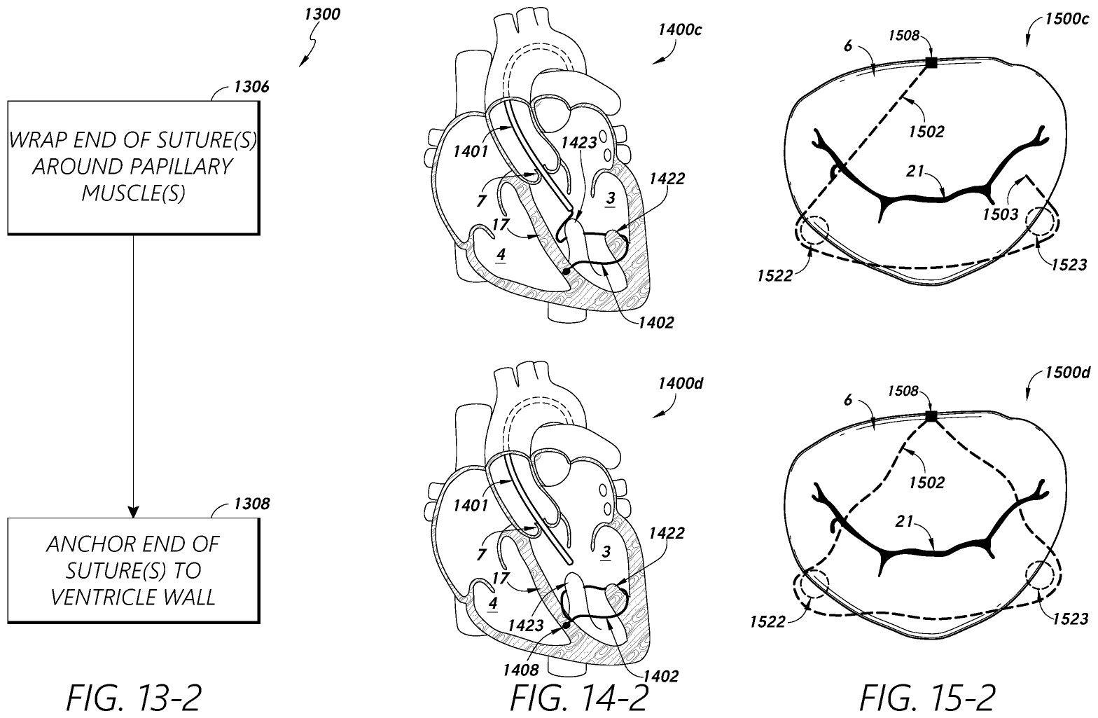

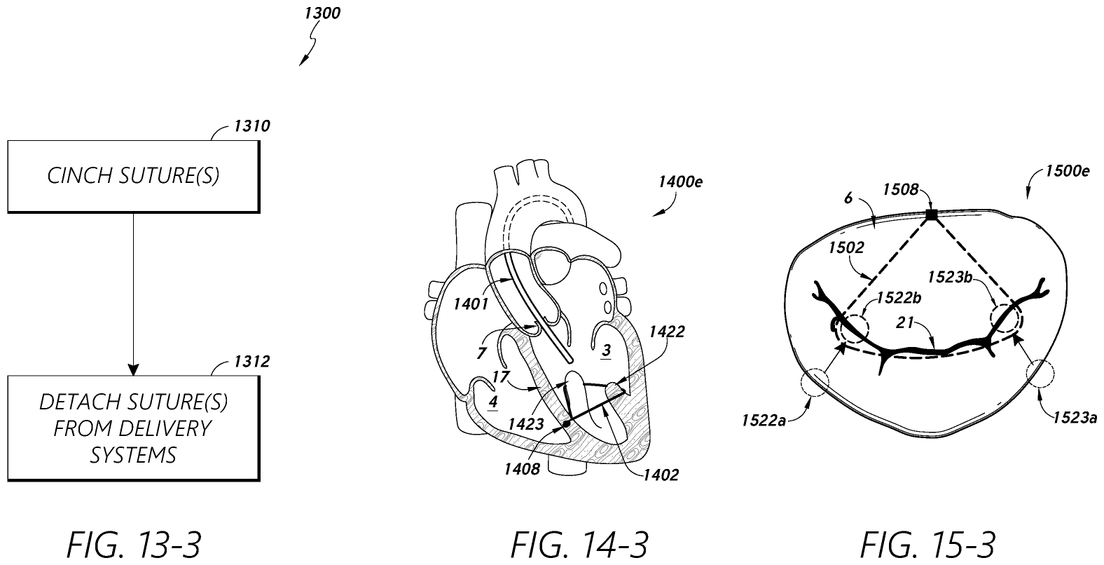

FIG. 13 is a flowchart for a transcatheter process 1300 of delivering a papillary muscle restoration device comprising a suture assembly to the heart. FIG. 14 provides several images corresponding to the process of FIG. 13, in which the restoration device is configured to be delivered via a transfemoral process. However, the restoration device may be configured to be delivered using a different method, such as a transapical process and/or a surgical process. FIG. 15 provides several overhead images of the mitral valve 6 and reference locations of the papillary muscles corresponding to various steps of the process 1300 of FIG. 13.

At block 1302, the process 1300 involves delivering one or more sutures of a suture assembly via a transcatheter and/or transfemoral procedure. As shown in image 1400a of FIG. 14, a catheter 1401 may be delivered via the aortic valve 7 to provide access to the left ventricle 3 for delivery of one or more sutures, including a first suture 1402. As shown in image 1500a of FIG. 15, the one or more sutures may be delivered while the heart is in a dilated state in which the papillary muscles 1522, 1523 are at dilated positions 1522a, 1523a, respectively, that are removed from target positions 1522b, 1523b, respectively. For example, the dilated position 1522a of the first papillary muscle 1522 and the dilated position 1523a of the second papillary muscle 1523 may be at least partially removed from being approximately directly below the coaptation line 21 of the mitral valve 6.

At block 1304, the process 1300 involves anchoring the one or more sutures to a ventricle wall (e.g., the septum 17, as shown in image 1400b). As shown in image 1500b of FIG. 15, a first end of a suture 1502 may be anchored to the ventricle wall via an anchoring element 1508 and a free end/portion 1503 of the suture may be exposed to the left ventricle 3 or other chamber of the heart.

At block 1306, the process 1300 involves wrapping a free end/portion of the suture around one or more papillary muscles. As shown in image 1400c of FIG. 14 and image 1500c of FIG. 15, the suture 1402, 1502 may be wrapped around two papillary muscles, a first papillary muscle 1422, 1522 and a second papillary muscle 1423, 1523. However, the one or more sutures 1402, 1502 may be wrapped around a single papillary muscle or around three papillary muscles. As the one or more sutures 1402, 1502 wrap around the one or more papillary muscles, the one or more sutures 1402, 1502 may contact an outer surface of the one or more of papillary muscles.

The one or more sutures 1402, 1502 may be configured to contact the papillary muscles at any point at the surface of the papillary muscle. In some embodiments, the suture 1402, 1502 may be configured to wrap around and/or contact a papillary muscle at or near a tip of the papillary muscle. However, to minimize risk of damage to the chordae tendineae connected to the papillary muscle, a suture 1402, 1502 may be wrapped around and or may be placed in contact with a portion of the papillary muscle that is near the tip of the papillary muscle while providing a sufficient distance from the chordae tendineae. Moreover, the one or more sutures 1402, 1502 may be anchored at any point along the ventricle wall. In some embodiments, the one or more sutures 1402, 1502 may be anchored at a point on the ventricle wall that is nearer to an apex region of the heart than to the mitral valve 6.

At block 1308, the process 1300 involves anchoring the free end/portion of the one or more sutures to the ventricle wall. In some embodiments, the free end/portion of the one or more sutures may be anchored at an anchoring element previously used to anchor the one or more sutures to the ventricle wall. For example, as shown in image 1400d of FIG. 14 and image 1500d of FIG. 15, multiple ends of the suture 1402, 1502 may be anchored to an anchoring mechanism 1408, 1508. However, the free end of the one or more sutures 1402, 1502 may be configured to be anchored using a different anchoring element and/or at a different point than the first anchoring point of the one or more sutures 1402, 1502.

By anchoring the free end of the one or more sutures 1402, 1502, the one or more sutures 1402, 1502 may form a loop around the one or more papillary muscles. As shown in image 1500d, in cases in which the free end of the one or more sutures 1402, 1502 is anchored at an anchoring point and/or anchoring mechanism 1508 previously used to anchor the one or more sutures 1402, 1502 to the ventricle wall, the one or more sutures 1402, 1502 may form a triangular shape having ends at the first papillary muscle 1522, the second papillary muscle 1523, and the anchoring point at the ventricle wall. The one or more sutures 1402, 1502 may have a single anchoring point at the wall or multiple anchoring points. In some embodiments, the one or more sutures 1402, 1502 may be configured to form a rectangular or other shape. For example, the one or more sutures 1402, 1502 may be configured to form a rectangular shape having corners at the first papillary muscle 1422, 1522, the second papillary muscle 1522, 1523, and at the two anchoring points at the ventricle wall.

At block 1310, the process 1300 involves cinching the one or more sutures to cause movement of the one or more papillary muscles. Cinching the one or more sutures may cause reduction the surface area of the loop created by the one or more sutures around the papillary muscles. Accordingly, as shown in image 1500e of FIG. 15, cinching the one or more sutures 1502 may cause a first papillary muscle 1522 to move from a dilated position 1522a to a restored position 1522b and/or may cause a second papillary muscle 1523 to move from a dilation position 1523a to a restored position 1523b. The restored positions 1522b, 1523b may be approximately below the coaptation line 21 of the mitral valve. Cinching the one or more sutures 1402, 1502, may cause the papillary muscles to move towards an anchoring point at the ventricle wall (e.g., the septum 17 as shown in image 1400e of FIG. 14).

At block 1312, the process 1300 involves detaching the one or more sutures from various delivery systems. For example, the suture 1402 and/or anchoring mechanism shown in image 1400e of FIG. 14 may be detached from the catheter 1401 and/or other devices.

While figures described herein may be described with reference to the heart and ventricle remodeling, some embodiments may be configured for delivery to parts of the body other than the heart and may be used for purposes other than ventricle remodeling. Moreover, while remodeling devices are shown as being implanted at one or more papillary muscles, optional embodiments may involve anchoring to multiple areas of ventricle walls and may not involve anchoring to the papillary muscles.

Depending on the embodiment, certain acts, events, or functions of any of the processes or algorithms described herein can be performed in a different sequence, may be added, merged, or left out altogether. Thus, in certain embodiments, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain embodiments require at least one of X, at least one of Y, and at least one of Z to each be present.

It should be appreciated that in the above description of embodiments, various features are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular embodiment herein can be applied to or used with any other embodiment(s). Further, no component, feature, step, or group of components, features, or steps are necessary or indispensable for each embodiment. Thus, it is intended that the scope of the inventions herein disclosed and claimed below should not be limited by the particular embodiments described above, but should be determined only by a fair reading of the claims that follow.

What is claimed is:

1. A method comprising:
anchoring a first suture to a first papillary muscle;
anchoring the first suture to a ventricle wall; and
cinching the first suture to cause the first papillary muscle to move towards the ventricle wall.

2. The method of claim 1, and further comprising anchoring the first suture to a second papillary muscle, wherein cinching the first suture causes the second papillary muscle to move towards the ventricle wall.

3. The method of claim 2, wherein cinching the first suture causes the first papillary muscle to move towards the second papillary muscle.

4. The method of claim 2, wherein a first end and a second end of the first suture are anchored to the ventricle wall, and anchoring the first suture to the first papillary muscle and the second papillary muscle involves wrapping the first suture at least partially around the first papillary muscle and the second papillary muscle.

5. The method of claim 1, and further comprising anchoring a second suture to the first papillary muscle.

6. The method of claim 5, wherein anchoring the first suture to the first papillary muscle involves attaching the first suture to the second suture.

7. The method of claim 6, and further comprising anchoring the second suture to a second papillary muscle.

8. The method of claim 6, wherein cinching the first suture involves pulling the second suture towards the ventricle wall.

9. The method of any of claim 1, and further comprising:
anchoring a second suture to a second papillary muscle;
anchoring the second suture to the ventricle wall; and
cinching the second suture, wherein cinching the second suture causes the second papillary muscle to move towards the ventricle wall.

10. The method of claim 9, wherein the first suture and the second suture at least partially overlap.

11. The method of claim 9, wherein the first suture is anchored to a first portion of the ventricle wall, the second suture is anchored to a second portion of the ventricle wall, and the first portion of the ventricle wall is closer to the second papillary muscle than the second portion of the ventricle wall.

12. A suture assembly comprising a first branch configured to anchor to a first papillary muscle, anchor to a ventricle wall, and cinch to cause the first papillary muscle to move towards the ventricle wall.

13. The suture assembly of claim 12, wherein the first branch is further configured to anchor to a second papillary muscle, and cinching the first branch further causes the second papillary muscle to move towards the ventricle wall.

14. The suture assembly of claim 13, wherein cinching the first branch further causes the first papillary muscle to move towards the second papillary muscle.

15. The suture assembly of claim 13, wherein a first end and a second end of the first branch are configured to be anchored to the ventricle wall, and anchoring the first branch to the first papillary muscle and the second papillary muscle involves wrapping the first branch at least partially around the first papillary muscle and the second papillary muscle.

16. The suture assembly of claim 12, and further comprising a second branch configured to anchor to the first papillary muscle.

17. The suture assembly of claim 16, wherein anchoring the first branch to the first papillary muscle involves attaching the first branch to the second branch.

18. The suture assembly of claim 17, wherein the second branch is configured to anchor to a second papillary muscle.

19. The suture assembly of claim 12, and further comprising a second branch configured to anchor to a second papillary muscle, anchor to the ventricle wall, and cinch to cause the second papillary muscle to move towards the ventricle wall.

20. A cardiac device comprising:
a suture assembly including a first branch; and
an anchoring element configured to anchor to a ventricle wall;
wherein the first branch is configured to attach to the anchoring element, anchor to a first papillary muscle, and cinch to cause the first papillary muscle to move towards the ventricle wall.
